(19) United States
(12) Patent Application Publication
(10) Pub. No.: US 2026/0091244 A1
(43) Pub. Date: Apr. 2, 2026

(54) THERAPEUTIC FACE MASK MODES OF OPERATION

(71) Applicant: SharkNinja Operating LLC, Needham, MA (US)

(72) Inventors: Sam William Bannister, Kent (GB); Christopher W. Hedges, Hampshire (GB); Christina J. Crowley, London (GB)

(21) Appl. No.: 18/901,465

(22) Filed: Sep. 30, 2024

(51) Int. Cl. A61N 5/06; A61N 5/00

(52) U.S. Cl. CPC A61N 5/0616; A61N 2005/007; A61N 2005/0647; A61N 2005/0652; A61N 2005/0659; A61N 2005/0663

(57) ABSTRACT

Various illustrative systems, devices, and methods for face masks are provided. In an exemplary implementation, a face mask is configured to be worn on a face of a user, includes a plurality of light emitting diodes (LEDs) configured to emit light toward and to skin on the face of the user with the user wearing the face mask, and is configured to operate selectively in a plurality of light therapy modes.

### FIELD

The present disclosure generally relates to face masks and more particularly to therapeutic face mask modes of operation.

### BACKGROUND

Light can be used to treat various skin conditions, particularly the skin on a user's face. Therefore, face masks with one or more light sources may be used to emit light towards the user's face. However, the light is generally very bright and is typically directed at the user's entire face. Such direct exposure to light can cause eye problems, such as eyesight problems so the user has to close their eyes in order to avoid point-blank exposure to light.

Different wavelengths of light are effective to treat different skin conditions. However, because of space restraints, excess cost, and/or other reason, some traditional face masks with light sources can only emit light of a particular color, such as red light or blue light. Such traditional face masks are thus limited in the treatments that the face mask can provide to a user.

Even traditional face masks that can emit light of different colors having different wavelengths often experience one or more problems. For example, face mask users generally expect light therapy treatment evenly on their face, but traditional face masks often fail to provide even light therapy. For another example, it is difficult for many users to select a particular light therapy treatment for the face mask to provide in each session of light therapy treatment. A similar problem in selecting cooling therapy exists for many users if the face mask can provide cooling therapy with or without also being able to provide light therapy. For yet another example, it is difficult for many users to keep accurate track of a number of times the face mask has provided therapy to the user.

Accordingly, there remains a need for improved devices, systems, and methods for face masks.

### SUMMARY

In general, systems, devices, and methods for face masks are provided.

In one aspect, a therapeutic device is provided that in one implementation includes a face mask configured to be worn on a face of a user. The face mask includes a plurality of light emitting diodes (LEDs) configured to emit light toward and to skin on the face of the user with the user wearing the face mask. Each of the plurality of LEDs is configured to emit first light having a first wavelength, second light having a second wavelength, and third light having a third wavelength. The face mask is configured to operate selectively in a plurality of light therapy modes. In a first light therapy mode of the plurality of light therapy modes, each of the LEDs is configured to emit the second light at a first power density, to emit the first light at a second power density, and to emit the third light at a third power density. In a second light therapy mode of the plurality of light therapy modes, each of the LEDs is configured to emit the second light having a fourth power density, to emit the first light at a fifth power density, and to emit the third light at a sixth power density. In a third light therapy mode of the plurality of light therapy modes, each of the LEDs is configured to emit the first light, to emit the second light having a seventh power density that is less than each of the first and fourth power densities, and to emit the third light at an eighth power density that is less than each of the third and sixth densities.

The therapeutic device can have any number of variations. For example, the seventh power density can be in a range of about 15% to about 35% of each of the first and fourth power densities, and the eighth power density can be in a range of about 70% to about 95% of each of the third and sixth power densities.

For another example, the third light therapy mode can be configured to be selected only after at least one of the first and second light therapy modes has been run a predetermined number of times. Further, the therapeutic device can also include a controller configured to track a number of times each of the first and second light therapy modes has been run. Further, the therapeutic device can also include a display configured to show the number of times to the user.

For yet another example, in the third light therapy mode, each of the LEDs can be configured to emit the first light at a ninth power density that is less than a maximum of the fifth power density. Further, the ninth power density can be in a range of about 85% to about 95% the maximum of the fifth power density.

For another example, the first light can be blue light, the second light can be red light, and the third light can be infrared (IR) light.

For yet another example, the first wavelength can be in a range of 450 nm to 495 nm, the second wavelength can be in a range of 620 nm to 740 nm, and the third wavelength can be in a range of 800 nm to 2500 nm.

For another example, the first light, the second light, and the third light are simultaneously emitted from each of the LEDs in each of the first, second, and third light therapy modes.

For still another example, a total power density of each of the lights in each of the first, second, and third light therapy modes can be about 128 mW/cm². Further, a total number of plurality lights can be 160.

For another example, each of the lights can be configured to be positioned a distance from the face of the user with the user wearing the face mask. Further, the distance can be in a range between about 10 mm and about 40 mm; the distance of each LED of the plurality of LEDs can be substantially equal for each LED, or the distance of at least one LED of the plurality of LED can be different from the distance of at least one other of the plurality of LEDs; and/or the face mask can also include a spacer configured to contact the face of the user with the user wearing the face mask, the spacer can include at least one of: a first spacer configured to contact a forehead of the face of the user, a second spacer configured to contact skin under eyes of the user, and a third spacer configured to contact skin over the eyes of the user. Further, the face mask can include at least one of the second spacer and the third spacer, and the at least one of the second spacer and the third spacer can be configured to block the light emitted by the plurality of LEDs from reaching the eyes of the user.

For yet another example, the face mask can also include a first thermoelectric cooling device configured to be positioned under a first eye of the user and configured to generate cooling configured to be applied to skin under the first eye of the user, and a second thermoelectric cooling device configured to be positioned under a second eye of the user and configured to generate cooling configured to be applied to skin under the second eye of the user. Further, the plurality of LEDs can be configured to emit the light simultaneously with the first and second thermoelectric cooling devices generating the cooling; and/or the plurality of LEDs can be configured to emit the light without the first and second thermoelectric cooling devices generating the cooling, and the first and second thermoelectric cooling devices can be configured to generate the cooling without the plurality of LEDs emitting the light.

For still another example, the therapeutic device can also include a control unit configured to allow the user to select which one of the plurality of light therapy modes to run.

For another example, the first light therapy mode can be a better aging mode, the second light therapy mode can be a skin clearing mode, and the third light therapy mode can be a skin sustain mode.

In another aspect, a method is provided that in one implementation includes using any of the therapeutic devices described above.

### BRIEF DESCRIPTION OF DRAWINGS

This disclosure will be more fully understood from the following detailed description taken in conjunction with the accompanying drawings, in which:

FIG. 1 is a perspective view of one implementation of a face mask and a control unit connected to the face mask with a cable;

FIG. 2 is a perspective view of the face mask and a portion of the cable of FIG. 1;

FIG. 3 is another perspective view of the face mask and a portion of the cable of FIG. 1;

FIG. 4 is another perspective view of the face mask and a portion of the cable of FIG. 1 with removable pads removed from the face mask;

FIG. 5 is a perspective view of the face mask and a portion of the cable of FIG. 1 omitting an inner shell of the face mask;

FIG. 6 is a perspective view of the face mask and a portion of the cable of FIG. 1 omitting the inner shell of the face mask and with removable pads removed from the face mask;

FIG. 7 is another perspective view of the face mask and a portion of the cable of FIG. 1 omitting the inner shell of the face mask and with removable pads removed from the face mask;

FIG. 8 is a perspective view of a portion of the face mask of FIG. 1 omitting an outer shell of the face mask;

FIG. 9 is another perspective view of a portion of the face mask of FIG. 1 omitting the outer shell of the face mask;

FIG. 10 is a perspective view of a portion of the face mask of FIG. 1 omitting non-removable pads of the face mask;

FIG. 11 is an exploded perspective view of a portion of the face mask of FIG. 1;

FIG. 12 is a schematic view of a positioning of pads of the face mask of FIG. 1 positioned under eyes of a user;

FIG. 13 is a perspective view of the control unit and a portion of the cable of FIG. 1;

FIG. 14 is another perspective view of the control unit and a portion of the cable of FIG. 1;

FIG. 15 is a side, partially cross-sectional view of the control unit and a portion of the cable of FIG. 1;

FIG. 16 is another perspective view of the control unit and a portion of the cable of FIG. 1;

FIG. 17 is a view of one implementation of a main menu screen of the control unit of FIG. 1;

FIG. 18 is a view of one implementation of a routines screen of the control unit of FIG. 1;

FIG. 19 is a view of the routines screen of FIG. 18 scrolled to the right;

FIG. 20 is a view of the routines screen of FIG. 19 scrolled to the right;

FIG. 21 is a view of the routines screen of FIG. 20 scrolled to the right;

FIG. 22 is a view of one implementation of a better aging start screen of the control unit of FIG. 1;

FIG. 23 is a view of one implementation of a better aging treatment screen of the control unit of FIG. 1;

FIG. 24 is a view of one implementation of a better aging end screen of the control unit of FIG. 1;

FIG. 25 is a view of one implementation of a cooling therapy duration selection screen of the control unit of FIG. 1;

FIG. 26 is a view of the routines screen of FIG. 25 with a different duration selected;

FIG. 27 is a view of one implementation of a cooling therapy screen of the control unit of FIG. 1;

FIG. 28 is a view of the cooling therapy screen of FIG. 27 with different options selected;

FIG. 29 is a view of the cooling therapy screen of FIG. 27 with different options selected;

FIG. 30 is a view of the cooling therapy screen of FIG. 27 with different options selected;

FIG. 31 is a view of the main menu screen of FIG. 17 with a progress option selected;

FIG. 32 is a view of one implementation of a progress screen of the control unit of FIG. 1;

FIG. 33 is a view of the main menu screen of FIG. 17 with a settings option selected;

FIG. 34 is a view of one implementation of a settings screen of the control unit of FIG. 1;

FIG. 35 is a perspective view of one implementation of a stand configured for use with the face mask of FIG. 1;

FIG. 36 is a perspective view of another implementation of a face mask;

FIG. 37 is another perspective view of the face mask of FIG. 36;

FIG. 38 is a perspective view of the face mask of FIG. 36 omitting an inner shell of the face mask;

FIG. 39 is another perspective view of the face mask of FIG. 36 omitting the inner shell of the face mask and omitting the non-removable pads of the face mask;

FIG. 40 is a schematic front view of lights of a mask positioned relative to a digital headform;

FIG. 41 is a schematic side view of the lights of FIG. 40 positioned relative to the digital headform;

FIG. 42 is a schematic view of the digital headform FIG. 40 with labeled facial regions;

FIG. 43 is a view of a simulation of an irradiation pattern of the lights of FIG. 40 on the digital headform;

FIG. 44 is a view of the simulation of FIG. 43 and simulations of irradiation patterns of the lights of FIG. 40 on smaller and larger digital headforms;

FIG. 45 is a front view of a simulation of an irradiation pattern of the lights of FIG. 40 on the digital headform with the lights misaligned relative to the digital headform; and

FIG. 46 is side view of the simulation of FIG. 45.

### DETAILED DESCRIPTION

Certain embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a value may not be precisely at a value but nevertheless be considered to be about that value or substantially at that value for any of a variety of reasons, such as sensitivity of measurement equipment or manufacturing tolerances.

Various illustrative systems, devices, and methods for face masks are provided. In general, a face mask is configured to provide cooling therapy and/or light therapy to a user wearing the face mask. The methods, systems, and devices described herein apply to face covering devices configured to provide light therapy and cooling therapy to a user wearing the face covering device, to face covering devices that are configured to provide cooling therapy without being configured to provide light therapy, and to face covering devices that are configured to provide light therapy without being configured to provide cooling therapy.

In an exemplary implementation in which a face covering device is configured to provide cooling therapy and light therapy, the face mask is configured to provide the cooling therapy and the light therapy simultaneously. Light cannot be felt by the user whereas the user will feel cooling, e.g., by feeling coolness at an area of the face adjacent the face mask's thermoelectric cooling device(s). Additionally, light therapy typically takes at least four weeks, and in some instances can take six to eight weeks or more, of daily mask use to provide a physical effect noticeable to the user, whereas cooling therapy typically provides a physical effect noticeable to the user after each session of receiving cooling therapy via the face mask. Cooling therapy may thus serve as an immediate physical signal to the user that the face mask is providing therapeutic effect while the user is wearing the mask and receiving cooling therapy and light therapy, e.g., because the user feels cooling, which may reassure that the face mask is working to provide treatment. Similarly, cooling therapy may serve as an immediate physical signal to the user that the face mask is providing therapeutic effect after each session of using the face mask, e.g., by noticing an effect in under-eye areas, whether or not light therapy is also being applied, which may improve user experience.

In an exemplary implementation in which the face covering device is configured to provide cooling therapy and light therapy to a user wearing the face covering device, the face covering device is also configured to, at a user's selection, provide cooling therapy and light therapy simultaneously or provide only one of cooling therapy and light therapy, thereby allowing for user-customized therapy. Over time a user's therapy needs may change, so the face mask being configured to provide user-customized therapy may extend the mask's useful life for a user. Additionally, different users may use the same mask, so the face mask being configured to provide user-customized therapy may allow each of the different users to select their own desired therapy, which may be cooling therapy only, light therapy only, or cooling therapy and light therapy.

Various exemplary implementations of face masks are described further in, for example, U.S. patent application Ser. No. 18/411,644 entitled “Face Masks With Noise Attenuation” filed Jan. 12, 2024, U.S. patent application Ser. No. 18/411,806 entitled “Light Emitting Face Masks” filed Jan. 12, 2024, and U.S. patent application Ser. No. 18/901,543 entitled “Face Masks With Therapeutic Cooling” filed on Sep. 30, 2024, which are hereby incorporated by reference in their entireties.

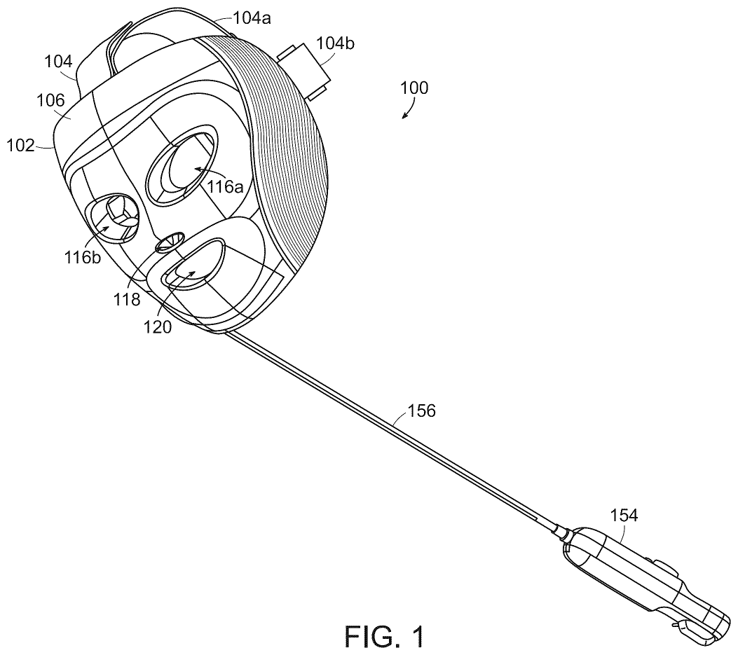

FIG. 1 illustrates one exemplary implementation of a face covering device (also referred to herein as a “face mask” or “mask”) 100 configured to provide cooling therapy and light therapy to a user wearing the face covering device 100. However, as mentioned above, in some implementations the face covering device 100 is not configured to provide light therapy or is not configured to provide cooling therapy.

The mask 100 includes a base 102 and a support 104 attached to the base 102. The base 102 is configured to be worn over a user's face. The support 104 is configured to be worn on the user's head to support the mask 100, and thus the base 102, on the user's head.

The support 104 can have a variety of configurations. For example, the support 104 can include a cap configured to be worn on a user's head similar to a hat. For another example, as in this illustrated implementation as shown in FIGS. 1-4, the support 104 can include a strap assembly including a first strap 104a and a second strap 104b attached to the first strap 104a. The first strap 104a is an upper portion of the strap assembly and is configured to be worn over and extend front-back along a crown of a user's head. The second strap 104b is a lower portion of the strap assembly and is configured to be worn around and extend substantially horizontally along a partial circumference of the user's head. The second strap's extension may not be precisely horizontal but nevertheless be considered to be substantially horizontal, depending on a particular user's head and how a user positions the second strap 104b. In an exemplary implementation, the first strap 104a and the second strap 104b are made from a flexible material, e.g., a textile, a plastic, or a combination thereof, which may help the first strap 104a and the second strap 104b comfortably conform to a size and shape of particular user's head.

In some implementations, the strap assembly includes padding, e.g., foam, air pockets, or other padding, configured to be positioned between the user's head and each of the strap assembly's straps to provide increased user comfort.

The strap assembly includes an adjustment mechanism, such as a buckle, snaps, Velcro, or other adjustment mechanism, configured to allow manual user adjustment of the first strap 104a and the second strap 104b to help fit the mask 100 snugly and comfortably on the user. The mask 100 in this illustrated implementation includes an adjustment mechanism in the form of Velcro (obscured in the figures) for adjustment of the first strap 104a and in the form of first and second sliders 104c, 104d for adjustment of left and right sides, respectively, of the second strap 104b. In other implementations, the strap assembly is self-adjusting, such as by straps of the strap assembly being elastic members similar to an elastic headband.

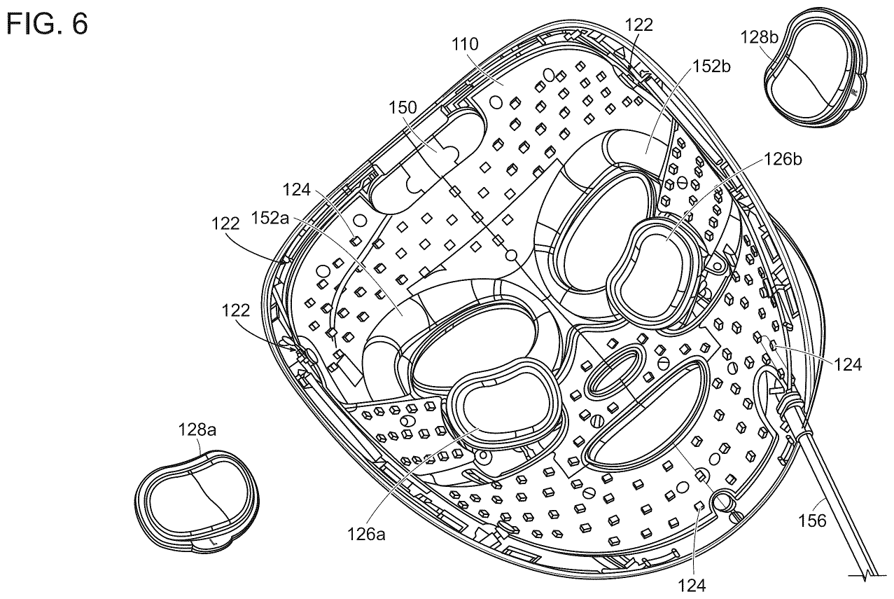

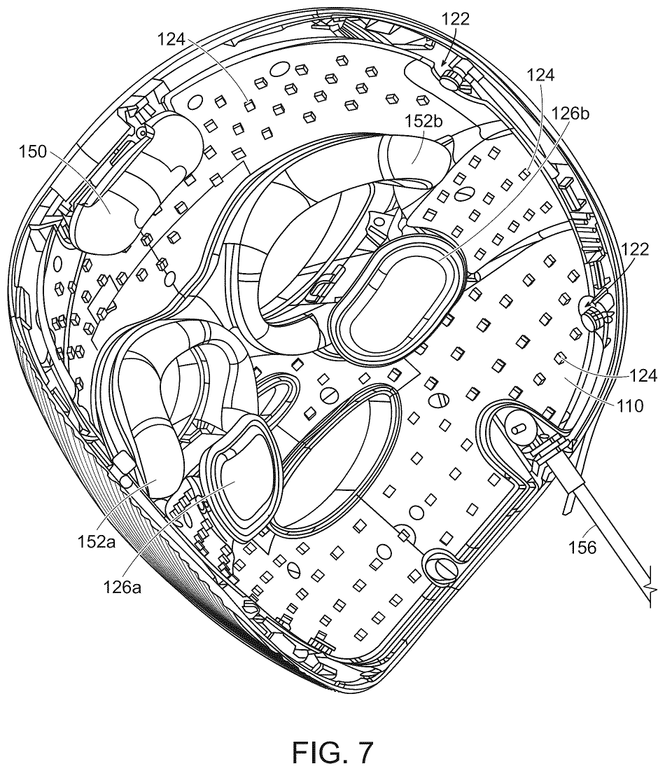

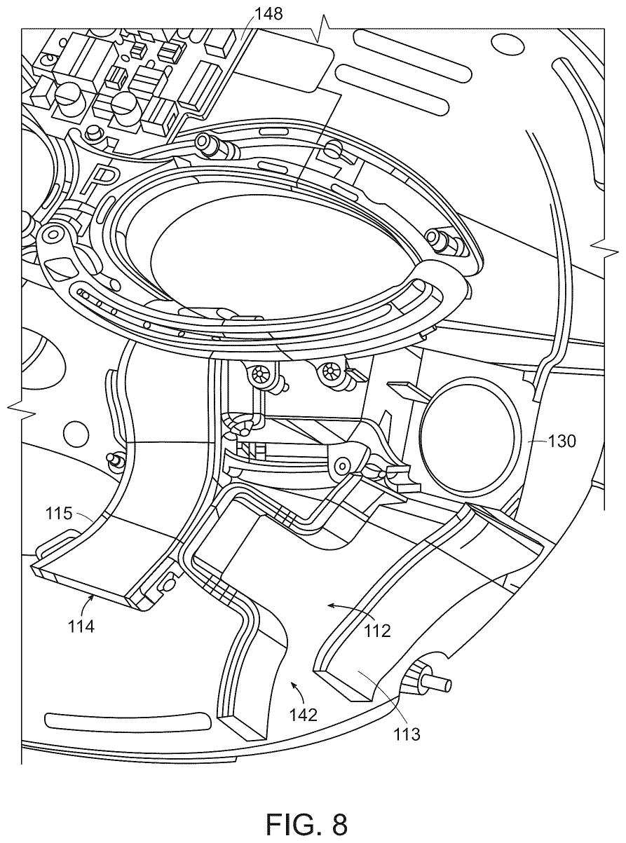

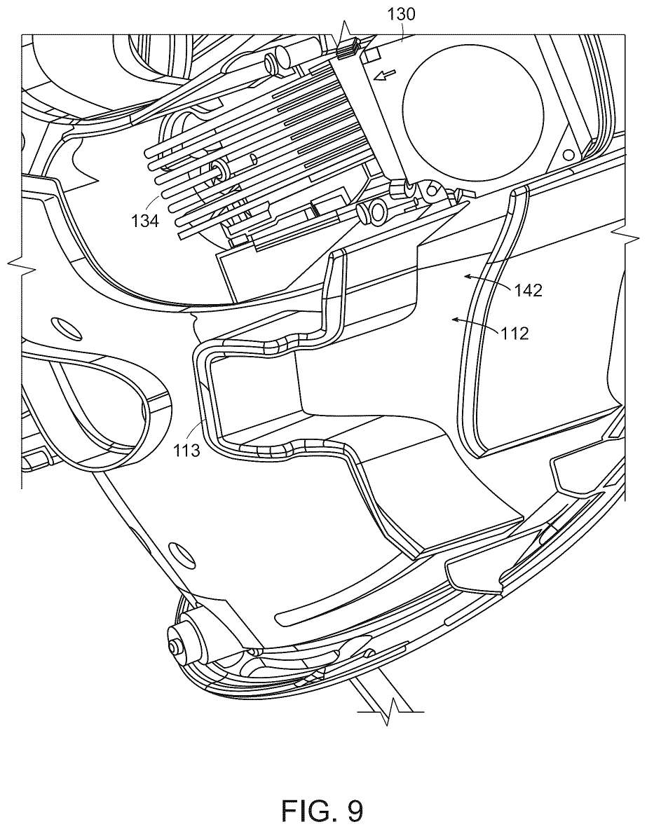

As shown in FIGS. 1-7, the base 102 includes an outer shell 106, an inner shell 108, and an intermediate shell 110 located between the outer and inner shells 106, 108. In an exemplary implementation, the base 102 is made from a rigid material, e.g., a plastic, a metal, or a combination thereof, which may help prevent the mask 100 from bending, deflecting, twisting, or otherwise breaking and/or may help prevent first and second air inflow paths 112 (see FIGS. 8 and 9) of the mask 100 and first and second air outflow paths 114 (see FIG. 9) of the mask 100, which are defined in the mask 100 by ducting 113, 115 (see FIGS. 8 and 9) formed between the outer and inner shells 106, 108, from deforming, twisting, or otherwise becoming at least partially obstructed so as to impede air flow. The second air inflow path and the second outflow path in the mask 100 on a right side of the mask 100 are obscured in the figures but are similar to the first air inflow path 112 and first air outflow path 114 on a left side of the mask 100.

The mask 100 includes a plurality of openings 116a, 116b, 118, 120 formed therein that each corresponds to a face feature and is configured to align at least partially with the face feature when the mask 100 is worn by a user. In this way, with a user wearing the mask 100, each of the plurality of openings 116a, 116b, 118, 120 will align at least partially with a feature of the user's face. Each of the plurality of openings 116a, 116b, 118, 120 is formed in the base 102 and is formed through all of the outer, inner, and intermediate shells 106, 108, 110. The plurality of openings include a first eye opening 116a configured to align at least partially with a right eye of a user wearing the mask 100, a second eye opening 116b configured to align at least partially with a left eye of the user wearing the mask 100, a nose opening 118 configured to align at least partially with a nose of the user wearing the mask 100, and a mouth opening 120 configured to align at least partially with a mouth of the user wearing the mask 100. The first and second eye openings 116a. 116b are configured to allow the user to see while wearing the mask 100 without the mask 100 preventing the user from being able to see anything except an inside surface of the mask 100, e.g., an inside surface of the inner shell 108. The nose opening 118 is configured to allow the user to easily use their nose, e.g., for breathing, etc., while the user is wearing the mask 100. The mouth opening 120 is configured to allow the user to easily use their mouth, e.g., for breathing, drinking, eating, etc., while the user is wearing the mask 100.

All of the plurality of openings 116a. 116b, 118, 120 in this illustrated implementation are unobstructed openings. In other implementations, one or more of the plurality of openings 116a. 116b, 118, 120 can be at least partially obstructed, such as with mesh, a transparent polymer plate, or other obstruction element.

The base 102 in this illustrated implementation is configured to cover substantially all of a user's face with the user wearing the mask 100. The base 102 may not entirely cover a particular user's face depending on a size and shape of the particular user's face, but the base 102 has a size configured to cover faces of most potential users of the mask 100. The base 102 thus includes openings 116a, 116b, 118, 120 for all of the user's eyes, nose, and mouth. In some implementations, the base 102 is configured to partially cover a user's face, such as only cover an upper half of a user's face, only cover a lower half of a user's face, cover a user's face except for left and right cheeks, or other partial coverage configuration. In such implementations, the base 102 may not have at least one of the eye, nose, and mouth openings 116a, 116b, 118, 120 depending on where the mask 100 is intended to be placed over a user's face. For example, whether or not the mask 100 is configured to provide light therapy, the mask 100 may not extend down to a user's mouth and thus not have the mouth opening 120 since, as discussed herein, cooling therapy is only applied under the user's eyes.

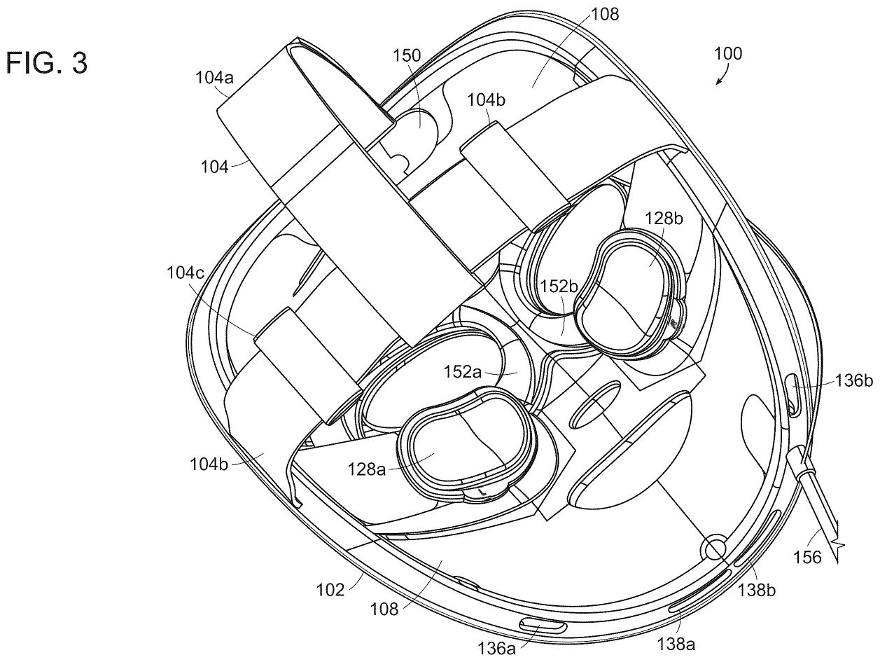

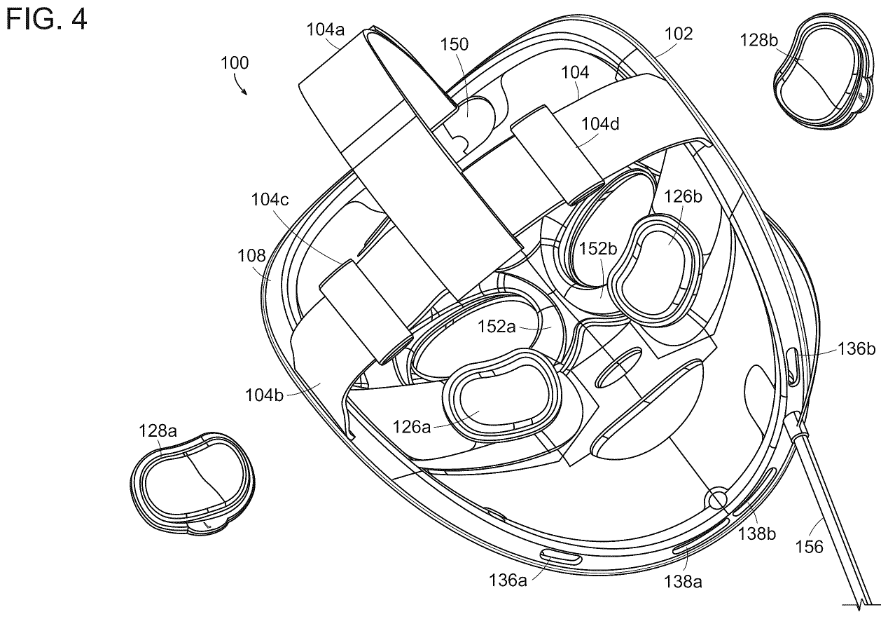

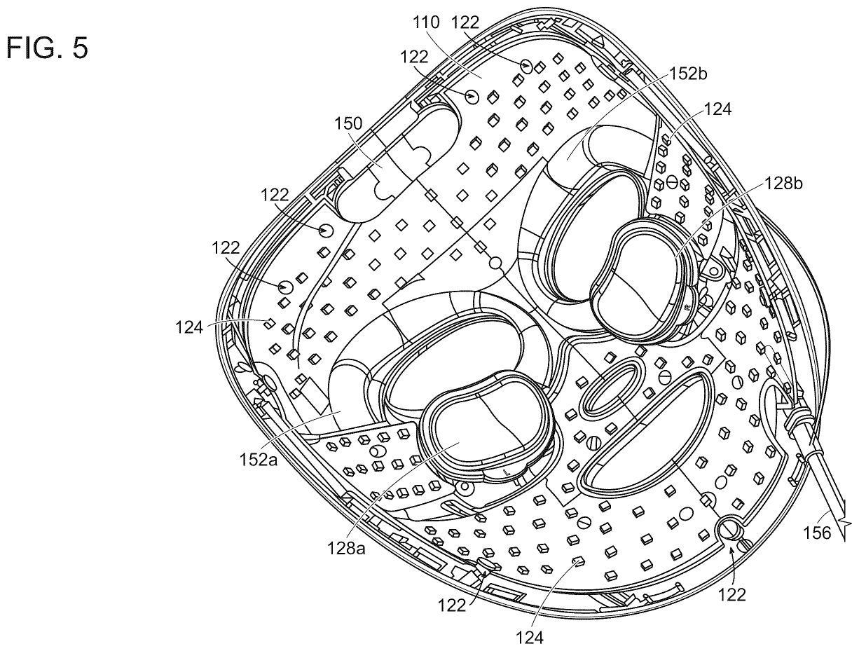

The outer shell 106 defines an exterior surface of the mask 100 that faces away from a user's face with the user wearing the mask 100. The inner shell 108 defines an interior surface of the mask 100 that faces toward a user's face with the user wearing the mask 100. The intermediate shell 110 is sandwiched between the outer and inner shells 106, 108. The mask 100 includes at least one interior connection point at which the outer, inner, and intermediate shells 106, 108, 110 are configured to be securely attached together, such as by using pins, adhesive, welding, etc. In this illustrated implementation, the outer and inner shells 106, 108 include protrusions that attach together through cut-outs 122 (see FIGS. 5-7) formed in the intermediate shell 110. The illustrated mask 100 includes nine interior connection points, but another number of interior connection points can be used.

A light assembly is located on the intermediate shell 110. The light assembly is configured to apply light therapy to a user wearing the mask 100. The light assembly includes a plurality of lights 124 spaced apart from one another in a pattern, e.g., a grid pattern, a random pattern, or other pattern, on the intermediate shell 110.

The light 124 are configured to be selectively turned on by a user, to provide the light therapy, and off by the user, to not be providing the light therapy. The lights 124 are light emitting diodes (LEDs) in this illustrated implementation. A number of the lights 124 can be, for example, in a range between ten and five hundred; in a range between two hundred and five hundred; in a range between three hundred and five hundred; in a range between four hundred and five hundred; in a range between ten and two hundred; in a range between fifty diodes and one hundred fifty; in a range between sixty and one hundred; in a range between seventy-five and eighty-five; in a range between two hundred and two hundred fifty; in a range between two hundred fifteen and two hundred twenty; eighty-five; fifty; seventy-five; eighty; eighty-five; two hundred, two hundred fifteen, two hundred twenty, two hundred forty, four hundred thirty, four hundred fifty, four hundred eighty, five hundred, or other number.

Each of the lights 124 is configured to emit light at at least one predetermined wavelength configured to facilitate various light therapies, such as one or more of an anti-aging treatment and an anti-breakout treatment. The predetermined wavelength can be, for example, a wavelength in a range between about 300 nm and about 1000 nm, or, for another example, in a range of about 450 nm to 2500 nm. The light emitted by the plurality of lights 124 is configured to reach one or more layers of skin, e.g., an epidermis, a dermis, and/or a hypodermis, of a user wearing the mask 100. The layer(s) of skin reached by the light corresponds to the wavelength. For example, a longer wavelength of light is configured to reach a deeper layer of skin than a shorter wavelength of light. In some implementations, the wavelength emitted by the lights 124 is configured to be adjustable, such as by one or more of a user manually selecting the wavelength and a wireless computing device configured to wirelessly communicate with a controller of the face covering device 100 to allow a user to manually select the wavelength.

In an exemplary implementation, each of the lights 124 is configured to emit red light, blue light, and infrared (IR) light. By being configured to emit light at different wavelengths (red wavelength, blue wavelength, and IR wavelength), energy from the lights 124 is configured to provide therapeutic benefit at different skin depths. IR light has a higher wavelength (e.g., in a range of 800 nm to 2500 nm) than blue light wavelength (e.g., in a range of 450 nm to 495 nm) and red light wavelength (e.g., in a range of 620 nm to 740 nm) so is configured to penetrate deeper into the skin than blue light and red light. The IR light energy emitted by the lights 124 is configured to be absorbed by skin, e.g., cells of skin, to provide one or more therapeutic benefits such as reducing deep wrinkles, preventing future fine lines, increasing collagen, and/or expanding blood vessels to allow for better blood flow. Red light has a higher wavelength than blue light so is configured to penetrate deeper into the skin than blue light. The red light energy emitted by the lights 124 is configured to be absorbed by skin, e.g., cells of skin, to provide one or more therapeutic benefits such as producing collagen to aid in anti-aging (e.g., smoothing wrinkles, increase skin firmness, reduce skin sagging, reducing fine lines, etc.). Blue light has a lower wavelength than red light and IR light and is configured to affect the skin's surface. The blue light energy emitted by the lights 124 is configured to be absorbed by skin, e.g., cells of skin, to provide one or more therapeutic benefits such as balancing skin texture and treating breakouts.

In some implementations, each of the lights 124 is configured to emit only red light. In some implementations, each of the lights 124 is configured to emit only blue light. In some implementations, each of the lights 124 is configured to emit only IR light. In some implementations, each of the lights 124 is configured to emit only red light and blue light. In some implementations, each of the lights 124 is configured to emit only blue light and IR light. In some implementations, each of the lights 124 is configured to emit only red light and IR light.

Various exemplary implementations of light assemblies for a face mask are described further in, for example, U.S. patent application Ser. No. 18/411,806 entitled “Light Emitting Face Masks” filed Jan. 12, 2024, which is hereby incorporated by reference in its entirety.

The lights 124 are configured to provide therapeutic light therapy. In some implementations, the mask 100 includes at least one light configured to emit light for a non-therapeutic purpose. For example, the mask 100 can include at least one light configured to illuminate when the mask 100 is powered on to assure a user that the mask 100 has been powered on. For another example, the mask 100 can include at least one light configured to illuminate during the mask's application of cooling therapy. Cooling therapy applied to a user's skin does not have a visual element detectable by a user, unlike light therapy which will typically be perceptible visibly to a user wearing the mask 100 since light is being directed toward the user's face, so the light(s) may assure the user that the cooling therapy is being provided. In an exemplary implementation in which the mask 100 is configured to provide at least cooling therapy, at least one light (e.g., a single light, a pair of lights, a row of five lights, a row of ten lights, etc.) is located under the left eye opening 116a for visualization by a left eye of a user wearing the mask 100 and at least one light is located under the right eye opening 116b for visualization by a right eye of the user wearing the mask 100.

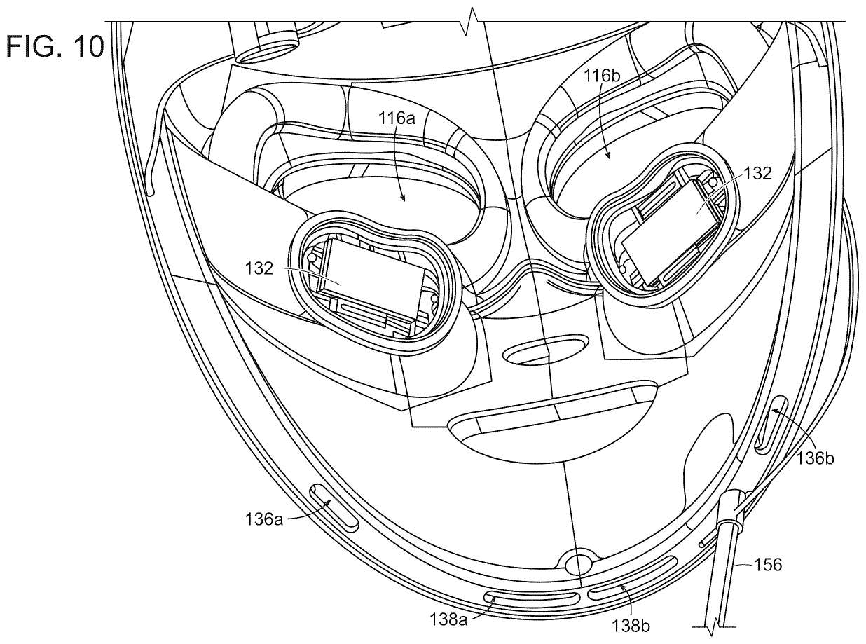

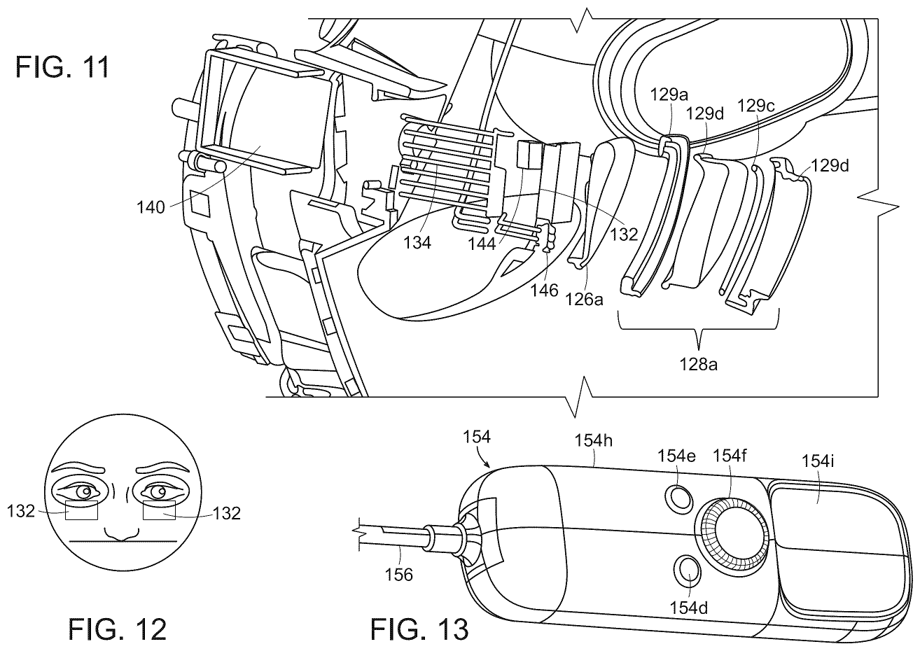

As mentioned above, the mask 100 is configured to provide cooling therapy to a user wearing the mask 100. As discussed further below, the mask 100 includes first and second thermoelectric cooling devices 132, as shown in FIGS. 10 and 11. Only one of the thermoelectric cooling devices 132 is shown in FIG. 11. The first thermoelectric cooling device 132 is associated with the first eye opening 116a of the mask 100 that is configured to align with a left eye of a user wearing the mask 100. The second thermoelectric cooling device 132 is associated with the second eye opening 116b of the mask 100 that is configured to align with a right eye of the user wearing the mask 100. The first and second thermoelectric cooling devices 132 are configured to be located below the user's left and right eyes, respectively, as shown for example in FIG. 12, to allow the cooling provided by the first and second thermoelectric cooling devices 132 to be applied below the user's left and right eyes. Cooling applied below the user's left and right eyes is configured to depuff, soothe, and refresh the user's under-eye area by shrinking blood vessels under the eyes.

The mask 100 includes a cooling system configured to provide cooling therapy to a user's under-eye area via a pair of pads configured to be positioned against skin under left and right eyes, respectively, of a user wearing the mask 100. In this illustrated implementation, the mask 100 is configured to be used with first and second sets of pads that each include two pads, one associated with each eye. In other implementations, the mask 100 can be configured to be used with only a single set of pads or with another plural number of sets of pads, e.g., three, four, etc.

The mask 100 is configured to be one-size-fits-all. However, because different people have different face shapes and contours, the first and second pads of one set of pads may feel more comfortable and/or have better contact with skin for some users than for other users. Better contact with skin more effectively provides the cooling effect to the user. The mask 100 in this illustrated implementation includes a plurality of sets of pads configured to be selectable by a user. The user may thus use the mask 100 with the set of pads that feels most comfortable and/or has the best skin contact. Additionally, by being usable with a selected set of pads, the same mask 100 may be more effectively used by different users as compared to a mask that does not include selectable pads.

FIGS. 4, 6, and 7 show the mask 100 with first and second pads 126a, 126b of the first set of pads attached to the mask 100 without alternate first and second pads 128a, 128b of the second set of pads attached to the mask 100. FIGS. 3 and 5 show the mask 100 with the alternate first and second pads 128a, 128b attached to the mask 100 and the first set of pads underlying the second set of pads, as discussed further below. FIG. 10 shows the mask 100 without either the first set of pads or the second set of pads attached to the mask 100 for purposes of illustration, since the first set of pads is non-removably attached to the mask 100 in this illustrated implementation.

The first set of pads is non-removably attached to the mask 100. By having a set of non-removable pads, the mask 100 prevents a user from using the mask 100 without pads, which may be uncomfortable and/or provide too much cooling of skin that could cause skin damage. The first and second pads 126a, 126b of the first set of pads thus define default pads of the mask 100.

The second set of pads is configured to be removably attached to the mask 100. The user may thus select whether to use the first and second pads 126a, 126b of first set of pads against the user's face, e.g., the second set of pads is not attached to the mask 100, or to use the alternate first and second pads 128a, 128 of the second set of pads against the user's face, e.g., the second set of pads is attached to the mask 100.

The second set of pads is configured to be removably attached to the mask 100 by clipping to the first set of pads. As shown in FIG. 11, which shows one of the pads 128a of the second set of pads as a representative example of both pads 128a, 128b of the second set of pads, the pad 128a include a lower clip 129a, a coolsink 129b, a compressive layer 129c, and an upper clip 129d. The lower and upper clips 129a, 129d are attached together to hold together the coolsink 129b and the compressive layer 129c.

FIG. 11 also shows one of the pads 126a of the first set of pads as a representative example of both pads 126a, 126b of the first set of pads. As shown in FIG. 11, the pad 126a includes a coolsink. Thus, with the second set of pads removably attached to the mask 100, two coolsinks are used together.

The second set of pads is configured to overlie the first set of pads with the second set of pads removably attached to the mask 100. The second set of pads is thus configured to extend farther beyond the mask 100 in a direction toward a user's face than the first set of pads and thus may feel more comfortable to some users than the first set of pads.

Each of the pads 126a, 126b of the first set of pads and the alternate pads 128a, 128b of the second set of pads has different curvature based on whether the pad is for positioning under a left eye or under a right eye, as a person's face has different curvatures under the eyes. The alternate pads 128a, 128b each include an identifier to indicate to a user where the alternate pads 128a, 128b should be attached to the mask 100, e.g., under the left eye opening 116a or under the right eye opening 116b. The identifiers in this illustrated implementation are the letters “L” (for left) and “R” (for right), but other identifiers can be used additionally or alternatively, such as the words “left” and “right,” the alternate left pad 128a being a first color that matches a color of the left pad 126a of the first set of pads and the alternate right pad 128b being a second color that is different than the first color that matches a color of the right pad 126b of the first set of pads, etc. The identifiers are on the upper clip 129d in this illustrated implementation but can be located elsewhere.

In some implementations, each of the first and second sets of pads is configured to removably attach to the mask 100, which may facilitate cleaning of the pads.

In some implementations, the mask 100 does not include any removable sets of pads. Instead, the mask 100 includes only a non-removable set of pads, which may help prevent loss of pads and/or effectively prevent a user from using the mask 100 without pads.

The mask's cooling system can have a variety of configurations. As in this illustrated implementation, as shown in FIGS. 8-11, the cooling system can include a fan 130, a thermoelectric cooling device 132, and a heat sink 134.

The mask 100 can include a single cooling system or, as in this illustrated implementation, can include a plurality of cooling systems. The illustrated mask 100 includes two cooling systems, a first cooling system (left-side cooling system) and a second cooling system (right-side cooling system). FIGS. 8, 9, and 11 show elements of the first cooling system. FIG. 10 shows elements of the first and second cooling systems. The first cooling system is associated with a left side of the mask 100 and thus with a left side of the user's face and the user's left eye when the mask 100 is on the user's face. The second cooling system is associated with a right side of the mask 100 and thus with a right side of a user's face and the user's right eye when the mask 100 is on the user's face. The mask 100 therefore includes first and second fans 130, first and second thermoelectric cooling devices 132, and first and second heat sinks 134. The second fan and the second heat sink are obscured in the figures. The first and second cooling systems are configured and used similarly so are not each particularly described, with features described for the first cooling system similarly applying to the second cooling system.

The thermoelectric cooling device 132 is configured to generate heat through a thermoelectric effect where a heat flux is created at a junction of two different types of materials. The heat flux creates a cold area and a hot area. The cold area is configured to face toward the user's face to provide cool energy to the user's skin at the user's face, e.g., via contact of either the first set of pads or the second set of pads with the user's skin. The hot area creates heat energy that the mask 100 is configured to dissipate to help prevent the heat from interfering adversely with the cooling provided to the user by the mask 100. Dissipating the heat generated from the thermoelectric cooling device 132 may help prevent the heat from interfering adversely with the cooling effect provided by the mask 100.

The thermoelectric cooling device 132 is a Peltier device in this illustrated implementation but can be another type of thermoelectric cooling device such as a thermoelectric cooler (TEC) or other thermoelectric cooling device. In this illustrated implementation, the Peltier device includes two ceramic plates with diodes located between the ceramic plates.

The illustrated fan 130 of the left-side cooling system is configured to blow air configured to dissipate the heat created by the thermoelectric cooling device 132 of the left-side cooling system. The mask 100 includes a first air inlet 136a through which air, e.g., ambient air, is configured to enter the mask 100. With the fan 130 on, the fan 130 is configured to draw the air into the mask 100 through the first air inlet 136a. The mask 100 includes an air inflow path along which the air is configured to flow to the fan 130 from the first air inlet 136a. The fan 130 is configured to blow the air across the thermoelectric cooling device 132, e.g., across the hot side of the thermoelectric cooling device 132, and then along an air outflow path of the mask 100 to a first air outlet 138a through which the air is configured to exit the mask 100. Air is thus configured to flow unidirectionally through the mask 100 from the first air inlet 136a to the first air outlet 138a. The mask 100 similarly includes, in relation to the right-side cooling system, a second air inlet 136b, a second air outlet 138b, a second air inflow path, and a second air outflow path.

The first and second air outlets 138a, 138b are shared air outlets in this illustrated implementation. Air entering the mask 100 through the first air inlet 136s is configured to exit out of either of the first and second air outlets 138a, 138b, and air entering the mask 100 through the second air inlet 136b is also configured to exit out of either of the first and second air outlets 138a, 138b. In some implementations, there is a different number of shared air outlets, e.g., one, three, etc. In some implementations, the mask 100 has separate air outlets for each of the mask's first and second air flow paths associated with the left and right sides of the mask 100 and thus with the left and right eyes of the user wearing the mask.

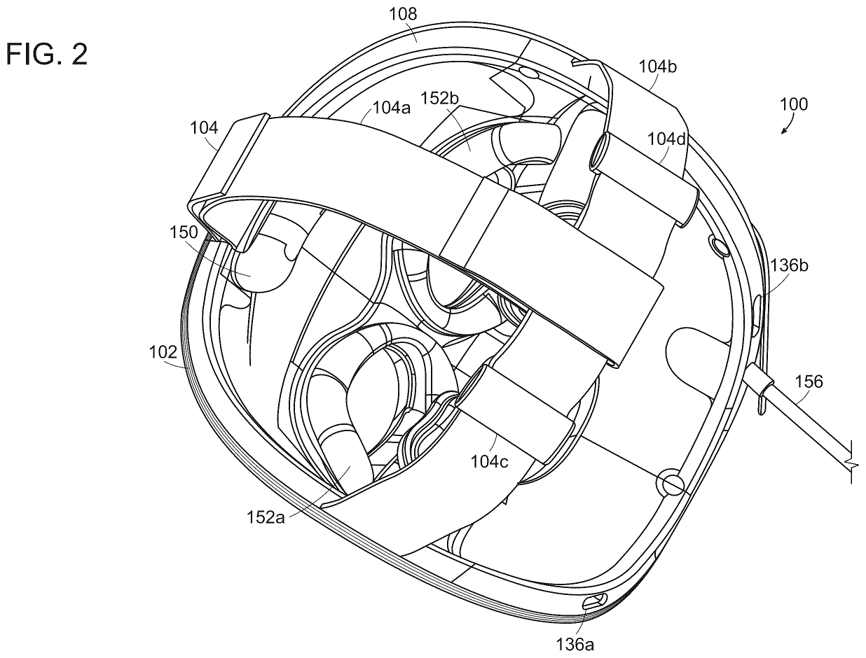

The first and second air inlets 136a, 136b and the first and second air outlets 138a, 138b are located at a bottom of the mask 100, as shown in FIGS. 2-4 and 10. Thus, as indicated by FIG. 1, the first and second air inlets 136a, 136b and the first and second air outlets 138a, 138b are not visible when looking at a front of the mask 100, which may improve aesthetics of the mask 100. In other implementations, any one or more of the first and second air inlets 136a, 136b and the first and second air outlets 138a, 138b can be located elsewhere, e.g., at a top of the mask 100 or a side of the mask 100, instead of at the bottom of the mask 100 and may thus similarly not be visible when looking at a front of the mask 100.

As mentioned above, the cold area of each of the thermoelectric cooling devices 132 is configured to face toward the user's face when the mask 100 is on the user face to provide cool energy to the user's skin, e.g., via contact of either the first set of pads or the second set of pads with the user's skin. The first and second pads 126a, 126b of the first set of pads are each a coolsink configured to transfer cooling from the first thermoelectric cooling device 132 to the user's skin (either directly or through the first and second pads 128a, 128b, which are also each a coolsink, of the second set of pads). The first and second sets of pads are metallic to facilitate the cold transfer, although another material is possible. Metal may more effectively transfer cold than other materials.

The hot area of each of the thermoelectric cooling devices 132 is configured to face away from the user's face, when the mask 100 is on the user face, to urge heat away from the user's skin. The first heat sink 134 faces the hot area of the first thermoelectric cooling device 132 to help the first heat sink 134 receive heat energy from the first thermoelectric cooling device 132, e.g., from the hot area thereof, to help prevent the heat from being applied to the user's face or interfering with the cooling effect provided to the user's skin via the first thermoelectric cooling device 132. In this illustrated implementation, the first heat sink 134 is made from aluminum but can have other configurations.

The first heat sink 134 is located downstream of the first fan 130. The first fan 130 is configured to blow air toward the first heat sink 134. The first fan 130 is thus configured to help dissipate the heat created by the first thermoelectric cooling device 132. Heat is therefore urged away from the first heat sink 134 and into a first cooling duct 140 that leads to the first air outflow path 114 to allow the air to exit out of the mask 100. The first fan 130 is operably coupled with the mask's controller, discussed further below, to allow the mask's controller to control the first fan 130, e.g., on/off status of the first fan 130, etc.

In some implementations, the first and second fans 130 are configured to stop running, e.g., under control of the controller at the PCB 148, when a cooling therapy session ends, which may conserve power and/or reduce an amount of time the user may hear generated noise that is radiated into the environment due to use of the first and second fans 130. In other implementations, the first and second fans 130 are configured to continue running, e.g., under control of the controller at the PCB 148, for a predetermined period of time, e.g., 10 seconds, 30 seconds, 45 seconds, 60 seconds, 65 seconds, 90 seconds, etc., after a cooling therapy session ends (with or without light therapy also having been provided in the session), which may help dissipate heat still in the mask 100 after the first and second thermoelectric cooling devices 132 have stopped generating heat.

The running of the first and second fans 130, e.g., when the fans 130 are on and causing air flow along the first and second air flow paths, creates noise that can irritate the user wearing the mask 100. The mask 100 includes first and second noise attenuation systems associated with the first and second fans 130, respectively. The first and second noise attenuation systems are configured to attenuate the generated noise that is radiated into the environment due to use of the first and second fans 130, respectively. The first and second noise attenuation systems are configured and used similarly so are not each particularly described, with features described for the first noise attenuation system similarly applying to the second noise attenuation system.

The first and second noise attenuation systems can have a variety of configurations. In this illustrated implementation, as shown in FIGS. 8-10, the first noise attenuation system includes a first acoustic chamber 142 upstream of its associated fan 130. The acoustic chamber 140 is generally configured as a silencer configured to muffle sound. The air entering the first air inlet 136a and flowing along the first air inflow path causes noise that would be easily heard by the user without the first acoustic chamber 140 being present to attenuate the noise so as to reduce, if not eliminate, bothersome noise to the user causes by the air inflow.

Various exemplary implementations of noise attenuation systems are described further in, for example, previously mentioned U.S. patent application Ser. No. 18/411,644 entitled “Face Masks With Noise Attenuation” filed Jan. 12, 2024, U.S. Pat. Pub. No. 2024/0257792 entitled “Acoustic Muffler For A Motorized Food Processing Device” published Aug. 1, 2024, and U.S. Pat. App. No. [ ] entitled “Face Masks With Therapeutic Cooling” [Attorney Docket No. 057664-793F01US] filed on even date herewith.

As shown in FIG. 11, the first cooling system of the mask 100 also includes a thermal cutoff (TCO) 144 and a temperature sensor 146. The temperature sensor 146 is a negative temperature coefficient (NTC) temperature sensor in this illustrated implementation but can be another type of temperature sensor. In general, the temperature sensor 146 and the TCO 144 are configured to help ensure that safe, comfortable, therapeutic cooling is provided to the user via the mask 100.

The temperature sensor 146 is configured to measure a temperature of the first coolsink 132 (first pad 132 of the first set of pads). The temperature sensor 146 is operatively coupled to a printed circuit board (PCB) 148 of the mask 100 (see FIG. 8) and is configured to provide the measured temperature to the PCB 148 of the mask 100.

The PCB 148 of the mask 100 includes electronic components (e.g., a controller, a memory, a bus, etc.) configured to facilitate operation of the mask 100. In this illustrated implementation the PCB 148 of the mask 100 is located in an upper portion of the mask 100 in a forehead area of the mask 100. Because of the relatively large amount of surface area in the forehead area of the mask 100, the PCB 148 being located in the upper portion of the mask 100 may allow for a larger, and thus more powerful, controller and other electronic components. In another implementation, the PCB 148 is located in a lower portion of the mask 100 in a chin area of the mask 100. The PCB 148 being in the lower portion of the mask 100 may ease manufacturing of the mask 100 for operable coupling of a control unit 154 and the PCB 148 of the mask 100 such as if a cable 156 extends from the lower portion of the mask 100. The control unit 154 and the cable 156 are discussed further below.

The TCO 144 of the mask 100 is configured as a safety device to interrupt electrical power to the first coolsink 132 in response to the temperature of the first coolsink 132 being above a maximum predetermined temperature. Interrupting the electrical power to the first coolsink 132 causes the first coolsink 132 to stop generating heat and cold. The first coolsink 132 will not typically reach a temperature above the maximum predetermined temperature, but in rare instance of malfunction, the TCO 144 may help prevent overheating of the first cooling system and/or an unsafe and/or uncomfortable temperature being provided against the user's skin.

Each of the first and second cooling systems including a temperature sensor and a TCO allows for independent control of the first and second cooling systems, which may improve accuracy of cooling therapy provided to the user.

Various exemplary implementations of face mask cooling therapy are described further in, for example, U.S. patent application Ser. No. 18/411,644 entitled “Face Masks With Noise Attenuation” filed Jan. 12, 2024 and U.S. patent application Ser. No. 18/901,543 entitled “Face Masks With Therapeutic Cooling” filed on Sep. 30, 2024, which are hereby incorporated by reference in their entireties.

As mentioned above, the first set of pads (or, alternatively, the second set of pads if removably attached to the mask 100) is configured to contact skin of a user wearing the mask 100. Other portions of the mask 100 are configured to contact skin of the user with the user wearing the mask 100. As shown in FIGS. 2-7, the mask 100 in this illustrated implementation includes a forehead pad 150 and first and second eye shields 152a, 152b. In other implementations, the forehead pad 150 and/or the first and second eye shields 152a, 152b are omitted.

The forehead pad 150 is configured to provide padding between the rigid base 102 and a forehead of a user wearing the mask 100 to improve user comfort and to help space the lights 124 a distance from the user's face. The forehead pad 150 is pill-shaped in this illustrated implementation but can have other shapes. The forehead pad 150 is a single pad, e.g., a single foam pad, in this illustrated implementation but can include multiple pads.

The first and second eye shields 152a, 152b are configured to provide padding between the rigid base 102 and areas around eyes of a user wearing the mask 100 to improve user comfort, to help space the lights 124 a distance from the user's face, and, when the mask 100 is providing light therapy, to help block light emitted from the lights 124 from distractingly shining into the user's eyes. The first and second eye shields 152a. 152b are each a single shield, e.g., a single silicone or other flexible member, in this illustrated implementation but can include multiple shields.

The first eye shield 152a extends around a portion of the first eye opening 116a and is thus associated with a left eye of a user wearing the mask 100. The first eye shield 152a extends around a partial perimeter of the first eye opening 116a to avoid interfering with cooling provided via the first cooling system, which is described further below. The second eye shield 152b extends around a portion of the second eye opening 116b and is thus associated with a right eye of a user wearing the mask 100. The second eye shield 152b extends around a partial perimeter of the second eye opening 116b to avoid interfering with cooling provided via the first cooling system, which is described further below.

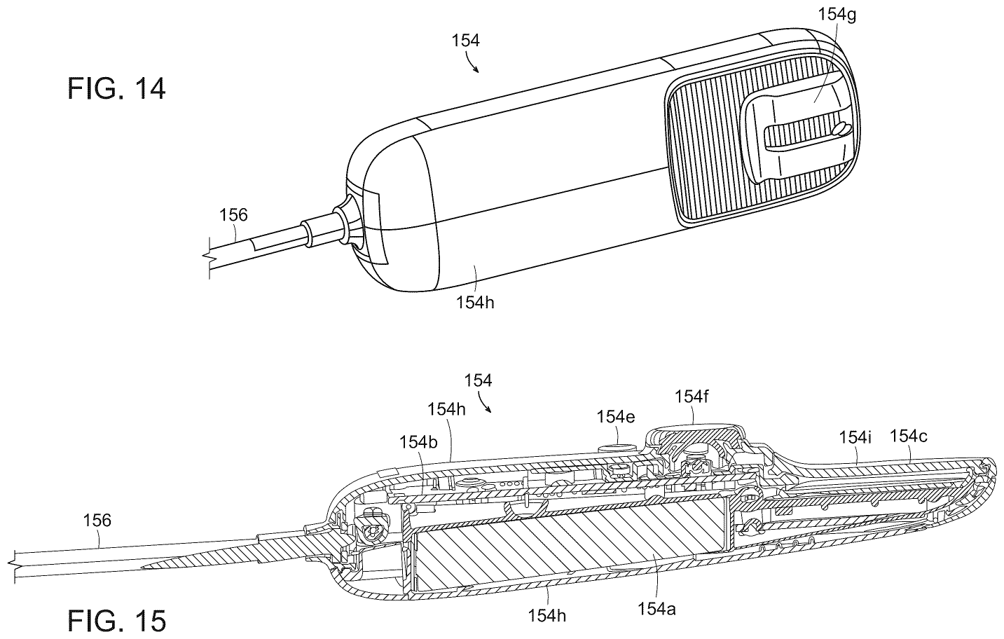

As shown in FIG. 1, a control unit 154 is connected to the mask 100. FIGS. 13-16 also show the control unit 154. The control unit 154 is configured to allow a user to control various functions of the mask 100. The control in this illustrated implementation includes control of the light therapy (e.g., turning the lights 124 on/off and adjusting light wavelength), control of the cooling therapy (e.g., turning cooling therapy on/off and adjusting cooling strength), control unit setting control (e.g., display brightness, display color scheme, settings reset, etc.), and power control (on/off).

The control unit 154 is connected to the mask 100 with a cable 156. FIG. 1 shows the cable 156 connected at a first end of the cable 156 to the control unit 154 and at a second end of the cable 156 to the mask 100. The cable 156 extends from a lower portion of the mask 100 in this illustrated implementation, which may help the cable 156 be as unobtrusive as possible while a user is wearing the mask 100. The cable 156 is fixedly attached to the mask 100 and the control unit 154, which may help prevent loss of one of the mask 100 and the control unit 154 and/or may help ensure proper electrical connection between the mask 100 and the control unit 154. In other implementations, the cable 156 can be detachable from one or both of the mask 100 and the control unit 154, which may facilitate cleaning of the mask 100 (e.g., if the cable 156 is detachable from the mask 100), may allow for the mask 100 and/or control unit 154 to be replaced due to damage or other reason (e.g., if the cable 156 is detachable from the damaged or otherwise undesirable mask 100 and/or control unit 154), and/or may allow for an upgraded control unit to be used with the mask 100 (e.g., if the cable 156 is detachable from the mask 100)

The control unit 154 in this illustrated implementation is wired and is a dedicated control unit for the mask 100 and thus cannot control other masks 100 or other devices. In other implementations, the control unit 154 is a dedicated control unit for the mask 100 but is a wireless remote control configured to connect wirelessly to the mask 100, e.g., via Bluetooth or other wireless communication protocol. The control unit 154 being configured to connect wirelessly to the mask 100 may improve user experience by not requiring a user wearing the mask 100 to hold or clip the control unit 154. In still other implementations, the control unit 154 is not a dedicated control unit for the mask 100 and can control other masks and/or other devices. Examples of non-dedicated control units include a mobile phone, a mobile tablet, and other computing devices configured to wirelessly communicate with the mask 100.

The control unit 154 includes a power source 154a, a PCB 154b including electronic components (e.g., a controller, a memory, a bus, etc.), a display 154c, a plurality of controls 154d, 154e, 154f, a clip 154g, and an outer housing 154h. In this illustrated implementation, a protective lens 154i is disposed over the display 154c to help protect the display 154c from scratches or other damage.

The power source 154a is configured to provide power to the PCB 154b of the control unit 154, to the lights 124 of the mask 100, and to the first and second cooling systems of the mask 100. The power source 154a includes a pair of rechargeable batteries in this illustrated implementation (only one of the batteries is visible in the view of FIG. 15), but another number of batteries or another power source can be used. The control unit 154 including the power source 154a instead of the mask 100 may help reduce a weight and/or bulkiness of the mask 100, which may provide for a better user experience. In other implementations, the mask 100 includes a power source instead of or in addition to the control unit 154.

The power source 154a in this illustrated implementation is attached to the PCB 154b via soldering, although another attachment mechanism can be used. Soldering the power source 154a to the PCB 154b may help reduce a profile of the control unit 154 because the power source 154a can be closer to the PCB 154b and the outer housing 154h, which may allow for a smaller control unit. A smaller control unit 154 may be lighter and easier for a user to hold or clip. A smaller control unit may allow for the control unit 154 to be less obtrusive during use of the mask 100, e.g., by more easily fitting in a user's pocket, by extending less from a user's body when clipped using the clip 154g, etc.

The plurality of controls 154d, 154e, 154f are configured to be actuated by a user to provide inputs to the control unit 154 for controlling various functionalities of the mask 100. The plurality of controls 154d, 154e, 154f in this illustrated implementation includes first, second, and third controls 154d, 154e, 154f. In other implementations, the control unit 154 includes a single control or a different plural number of controls, e.g., two, four, five, etc.

The first control 154d is a cooling button configured to be actuated by a user, e.g., pressed down, to turn cooling therapy on and off. In other implementations, another type of cooling control can be used, such as a toggle switch, a lever, or other control.

The second control 154e is a back button configured to be actuated by a user, e.g., pressed down, to move back to an immediately previous screen shown on the display 154c. In other implementations, another type of back control can be used, such as a toggle switch, a knob, or other control.

The third control 154f is a knob configured to be actuated by a user in a first motion, e.g., rotated, to scroll through options shown on the display 154c, to be actuated by a user in a second motion, e.g., pushed down for less than a predetermined amount of time, to select an option shown on the display 154c or to pause a running therapy mode, and to be actuated by a user in a third motion, e.g., pushed down for at least the predetermined amount of time, to turn the control unit 154 on or off. In other implementations, one or more other type of controls can be used for scrolling through options, for selecting an option, and for power on/off, such as a toggle switch, lever, or other control.

The plurality of controls 154d, 154e, 154f are each operatively coupled to the PCB 154b of the control unit 154. In response to one of the plurality of controls 154d, 154e, 154f being actuated, the PCB 154b of the control unit 154 is configured to control the control unit 154 to cause the selected action to occur, e.g., power the control unit 154 on or off, show different information on the display 154c, cause cooling therapy to begin, cause light therapy to begin, reset control unit settings, etc.

The display 154c can be any of a variety of types of displays, such as a liquid crystal display (LCD) display or other type of display. In some implementations, instead of or in addition to the control unit 154 including the control(s) configured to be actuated by a user to provide inputs to the control unit 154, the display 154c is a touchscreen configured to receive input from a user.

The display 154c is configured to show information to the user regarding the mask 100. The information shown on the display 154c can include, for example, a power status (on/off), a battery charge level indication, a selected light therapy mode (also referred to herein as “light modes”), a selected cooling therapy mode (also referred to herein as “cooling modes”), an error status (e.g., an error code, a message explaining an error encountered, etc.), stored settings, stored historical use data (e.g., number of times and/or dates light therapy has been delivered from the mask 100, number of times and/or dates cooling therapy has been delivered from the mask 100, etc.), current date, current time, help information regarding use of the mask 100 and/or the control unit 154, etc.

The clip 154g of the control unit 154 is configured to allow the control unit 154 to be clipped to an item at a convenient location, e.g., a pocket, a collar, a shirt cuff, a waistband, a blanket, etc., so a user wearing the mask 100 does not have to hold the control unit 154 by hand while the mask 100 is providing cooling therapy and/or light therapy. Cooling therapy and light therapy each typically last at least a few minutes (e.g., in a range of about four minutes to about fifteen minutes), so user experience may be improved by the user not having to hold the control unit 154 by hand throughout the light therapy and/or cooling therapy.

The clip 154g is located on a back side of the control unit 154 that is opposite to a front side of the control unit 154 where the plurality of controls 154d, 154e, 154f and the display 154c are located. The plurality of controls 154d, 154e, 154f are thus configured to be easily accessible and the display 154c is configured to be easily visible when the control unit 154 is clipped to clothing or other item with the clip 154g. The clip 154g can, however, be located elsewhere.

The illustrated control unit 154 does not include any lights, but in other implementations the control unit 154 can include one or more lights configured to provide information to a user. For example, a light being on can indicate that power is on and the light being off can indicate that power is off. For another example, a light being on can indicate that cooling therapy is being provided and the light being off can indicate that cooling therapy is not being provided. For yet another example, a light being on can indicate that light therapy is being provided and the light being off can indicate that light therapy is not being provided.

The control unit 154, e.g., the controller of the control unit's PCB 154b, is configured to be in operable communication with the mask 100, e.g., the controller of the mask's PCB 148. In this illustrated implementation, each of the control unit 154 and the mask 100 includes a PCB 154b, 148. In some implementations, only one of the control unit 154 and the mask 100 includes a PCB, which may reduce cost and/or ease manufacturing.

In an exemplary implementation, the control unit 154 is configured to allow a user to select one of a plurality of menu options, e.g., by using the third control 154f to scroll through available menu options shown on the display 154c and select a desired one of the menu options by pushing the third control 154f down for less than the predetermined amount of time. FIG. 17 illustrates one implementation of a main menu screen 160 configured to be shown on the display 154c. The main menu screen 160 shows the plurality of menu options. All of the menu options are visible on the main menu screen 160 in this illustrated implementation. If all of the menu options are not visible simultaneously, the user can scroll through the menu options, e.g., by using the third control 154f, to see all the available menu options.

The main menu screen 160 is a default screen configured to be shown on the display 154c when the control unit 154 is powered on. However, a first time the control unit 154 is turned on a help screen can be shown on the display 154c instead of the menu screen 160 to allow a user to view tutorial information (e.g., text, still images, and/or video) to become familiar with use of the mask 100 and the control unit 154 before selecting or beginning any cooling therapy or light therapy. As discussed further below, tutorial information is configured to always be available to the user via the main menu screen 160.

As in this illustrated implementation, the menu options on the main menu screen 160 can include a routines option, a progress option, and a settings option. The routines option allows a user to select one of a plurality of therapeutic modes (“modes” are also referred to herein as “modes of operation”), e.g., by using the third control 154f to scroll through available therapeutic modes shown on the display 154c and select a desired one of the therapeutic modes. The plurality of therapeutic modes include a plurality of light modes and a plurality of cooling modes. The plurality of light modes correspond to different light therapies. The plurality of cooling modes correspond to different cooling therapies.

FIGS. 18-21 illustrate one implementation of a routines screen 162 configured to be shown on the display 154c and to show the plurality of therapeutic modes in response to user selection of the routines option. As in this illustrated implementation, the plurality of therapeutic modes can include a better aging option, a skin clearing option, a skin sustain option, and an under-eye revive option. In some implementations, all of the plurality of therapeutic modes are visible on the routines screen 162. In this illustrated implementation, all of the plurality of therapeutic modes are not visible simultaneously on the routines screen 162. The routines screen 162 is configured to allow the user to scroll through the plurality of therapeutic modes, e.g., by using the third control 154f, to see all the available plurality of therapeutic modes. As the user scrolls through the routines screen 162, the plurality of therapeutic modes are each viewable, as shown by the series of routines screen 162 views in FIGS. 18-21.

As in this illustrated implementation, each of the plurality of therapeutic modes can be described on the routines screen 162 to help a user select one of the plurality of therapeutic modes.

The better aging option, the skin clearing option, and the skin sustain option correspond to different lights modes of light therapy. The under-eye revive option corresponds to cooling therapy and, when selected, allows the user to select a cooling mode.

The better aging option (also referred to herein as a “rehabilitation option”) allows selection of a better aging mode, which is a skin treatment mode for smoothing wrinkles and tightening sagging skin, as indicated on the routines screen 162 to help a user understand why this option may be desirable to the user. The better aging mode is configured to run for about six minutes, as also indicated on the routines screen 162 (see FIG. 18). A user is thus aware how long the better aging light therapy will last and thus how long the mask 100 must be worn for this treatment session.

In the better aging mode, each of the lights 124 of the mask 100 emits red light and IR light and does not emit blue light. Power density of the lights 124 in the better aging mode, and the other light modes, affects effectiveness of the light therapy. In an exemplary implementation, a power density ratio of the red light to the IR light is substantially 1:1, e.g., about 50% red light and about 50% IR light (blue light at zero), and the power density of a single light 124 in the better aging mode is about 64.0 mW/cm² for red light and about 64.0 mW/cm² for IR light for a total power density of each of the lights 124 in the better aging mode is thus about 128 mW/cm² and a total energy directed into the user face being about 215.7 J for each of red light and IR light for a total energy directed into the user face for thermal purposes being about 431.4 J for each light 124.

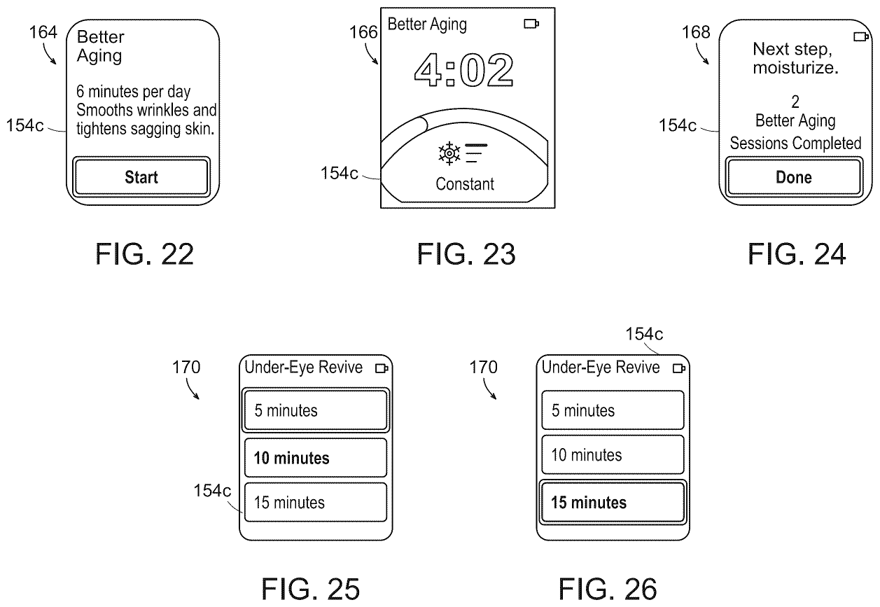

Selection of the better aging option via the routines screen 162, e.g., by pushing the third control 154f down for less than the predetermined amount of time with the better aging option shown on the routines screen 162 as in FIG. 18, is configured to show a better aging start screen 164 on the display 154c, as shown in FIG. 22. When the user is ready to begin the better aging treatment, the user selects to start the better aging mode, e.g., start the lights 124 emitting red light and IR light, via the better aging start screen 164, e.g., by pushing the third control 154f down for less than the predetermined amount of time.

Starting the better aging mode is configured to cause a better aging treatment screen 166 to be shown on the display 154c, as shown in FIG. 23. The better aging treatment screen 166 is configured to provide runtime information to the user regarding the running better aging treatment. As in this illustrated implementation, the information can include a type of the running light treatment (“better aging”) and a time indicator for the running light treatment. The better aging treatment screen 166 shows the time indicator as a time remaining for the running treatment with a clock countdown (FIG. 23 shows 4 minutes and 2 seconds remaining in this example) and with a progress bar (an arced bar in this example). Instead of or in addition to showing the time indicator being the time remaining for the running treatment, the time indicator can be time elapsed for the running treatment. In some implementations, the settings option accessible via the main menu screen 160 can allow the user to select whether to see time remaining for the running treatment, time elapsed for the running treatment, or both.

Cooling therapy is configured to begin automatically with the start of the better aging treatment. The cooling therapy beginning automatically may maximize mask 100 use since the user is already wearing the mask 100 to receive light therapy and/or may serve as an immediate physical signal to the user that the mask 100 is providing therapeutic effect while the user is wearing the mask 100 and receiving cooling therapy and light therapy.

As shown in FIG. 23, the better aging treatment screen 166 is configured to provide runtime information to the user regarding the running cooling treatment. As in this illustrated implementation, the running cooling treatment information can include a temperature level of the cooling treatment and the cooling mode. The temperature levels are shown in this illustrated implementation with graduated bars, with the longest bar indicating highest temperature and the shortest bar indicating lowest temperature. Three temperature levels are available in this illustrated implementation, although more than or less than three temperature levels may be provided, as discussed above. The second (middle) temperature level is shown in FIG. 23 as being run. The cooling mode is shown in this illustrated implementation with a text indicator. The cooling mode is “constant” mode in this example, indicating that cooling at the second temperature level is being provided constantly by the mask 100.

At any time during the better aging treatment, the user can choose, via the control unit 154, to turn off the cooling treatment, to change the temperature level, or to change the cooling mode. For example, actuating the first control 154d, pushing down the cooling button, is configured to scroll through the various cooling options. Each actuation of the first control 154d is configured to scroll through the different cooling options, e.g., each of the different temperature levels (including no temperature level for cooling treatment being “off”) and each of the different cooling modes. The better aging treatment screen 166 remains on the display 154c so the user can still see the better aging runtime information while adjusting the cooling treatment. The time indicator on the better aging treatment screen 166 may, for example, help a user decide when and whether to adjust cooling temperature level up or down for a remainder of the better aging treatment.

At an end of the better aging treatment, e.g., after the about 6 minutes have elapsed, the display 154c is configured to show a better aging end screen 168, as shown in FIG. 24. The better aging end screen 166 is configured to provide post-treatment information to the user regarding the better aging treatment that was just completed. As in this illustrated implementation, the post-treatment information can include one or more actions for the user to perform to help maximize beneficial effects of the better aging treatment, which in this example is to moisturize the face, and can include a total number of better aging treatment sessions that have been completed thus far, two in this example. The user being aware of a total number of completed treatment sessions may help a user decide when to select the skin sustain option (discussed further below) and/or may help encourage the user by showing a higher number with each treatment.

The better aging end screen 168 is configured to allow the user to go back to the main menu screen 160, e.g., by pushing the third control 154f down for less than the predetermined amount of time.

The skin clearing option allows selection of a skin clearing mode, which is a skin treatment mode for eliminating acne-causing bacteria, as indicated on the routines screen 162 (see FIG. 19) to help a user understand why this option may be desirable to the user. The skin cleaning mode also soothes red, puffy skin to give a clearer and calmer complexion. The skin clearing mode is configured to run for about eleven minutes, as also indicated on the routines screen 162 (see FIG. 19). A user is thus aware how long the skin clearing light therapy will last and thus how long the mask 100 must be worn for this treatment session.

In the skin clearing mode, the lights 124 of the mask 100 emit all three available types of light (red, blue, and IR) over the course of a skin clearing treatment session but emit only two of the light types at a time depending on a stage of the skin clearing treatment session. Each of the lights 124 emits a same two of the light types. The two types of light are different in each of the plurality stages, which allows different layers of skin to be treated in the skin clearing treatment session. In an exemplary implementation, a total power density of each light 124 is the same in each of the plurality of stages but a split of the total power density between the different light types varies between the different stages to better effectuate different aspects of treatment in the different stages.

In a first stage of the skin clearing treatment session, the lights 124 emit blue light and IR light and do not emit red light. In an exemplary implementation, a power density ratio of the blue light to the IR light is substantially 1:1, e.g., about 50% blue light and about 50% IR light (red light at zero), and the power density of a single light 124 in the first stage of the skin clearing mode is about 64.0 mW/cm² for blue light and about 64.0 mW/cm² for IR light for a total power density of each of the lights 124 in the first stage of the skin clearing mode is thus about 128 mW/cm², and a total energy directed into the user face being about 27.1 J for each of blue light and IR light for a total energy directed into the user face for anti-bacterial purposes being about 27.1 J for each light 124 and a total energy directed into the user face for thermal purposes being about 27.1 J for each light 124.

In a second stage of the skin clearing treatment session that follows the first stage, the lights 124 emit blue light and red light and do not emit IR light. In an exemplary implementation, a power density ratio of the blue light to the red light is substantially 0.75:1. e.g., about 43% blue light and about 57% red light (IR light at zero), and the power density of a single light 124 in the second stage of the skin clearing mode is about 55.0 mW/cm² for blue light and about 73.0 mW/cm² for red light for a total power density of each of the lights 124 in the second stage of the skin clearing mode is thus about 128 mW/cm², and a total energy directed into the user face being about 110.2 J for blue light and about 146.2 J for red light for a total energy directed into the user face for anti-bacterial purposes being about 110.2 J for each light 124 and a total energy directed into the user face for thermal purposes being about 146.2 J for each light 124. In other words, the power density of the blue light is about 75% of the power density of the red light.

In a third stage of the skin clearing treatment session that follows the second stage, the lights 124 emit IR light and red light and do not emit blue light. In an exemplary implementation, a power density ratio of the IR light to the red light is substantially 0.75:1, e.g., about 43% IR light and about 57% red light (blue light at zero), and the power density of a single light 124 in the second stage of the skin clearing mode is about 55.0 mW/cm² for IR light and about 73.0 mW/cm² for red light for a total power density of each of the lights 124 in the third stage of the skin clearing mode is thus about 128 mW/cm², and a total energy directed into the user face being about 110.9 J for IR light and about 147.2 J for red light for a total energy directed into the user face for anti-bacterial purposes being about zero J for each light 124 and a total energy directed into the user face for thermal purposes being about 258.1 J for each light 124. In other words, the power density of the IR light is about 75% of the power density of the red light.

The control unit 154 is configured to automatically move through the plurality of stages during the skin clearing treatment session. It would be difficult for a user to accurately manually change between the stages during the skin clearing treatment session. Thus, in the example above, the control unit 154 is configured to automatically move from the first stage to the second stage and from the second stage to the third stage during the skin clearing treatment session. In an exemplary implementation, the second and third stages each last substantially the same amount of time and the first stage lasts about 20% to about 25% of the time of each of the second and third stages, e.g., about 20%, about 21%, about 22%, etc. For example, for a skin clearing treatment session lasting a total of about 8 minutes, the first stage can last about 0.8 minutes and each of the second and third stages can last about 3.8 minutes. For another example, for a skin clearing treatment session lasting a total of about 11 minutes, the first stage can last about 1.4 minutes and each of the second and third stages can last about 4.8 minutes.

Selection of the skin clearing option via the routines screen 162, e.g., by pushing the third control 154f down for less than the predetermined amount of time with the skin clearing option shown on the routines screen 162 as in FIG. 19, is configured to show a skin clearing start screen on the display 154c. The skin clearing start screen is configured and used similar to the better aging start screen 164 discussed above. When the user is ready to begin the skin clearing treatment, the user selects to start the skin clearing mode, e.g., start first stage of the skin clearing treatment (which, in some implementations, may be the only stage if only one stage is provided instead of a plurality of stages), via the skin clearing start screen.

Starting the skin clearing mode is configured to cause a skin clearing treatment screen to be shown on the display 154c. The skin clearing treatment screen is configured and used similar to the better aging treatment screen 166 discussed above, e.g., provide runtime information to the user regarding the running skin clearing treatment.

Cooling therapy is configured to begin automatically with the start of the skin clearing treatment, similar to that discussed above regarding the better aging treatment. The skin clearing treatment screen is configured to provide runtime information to the user regarding the running cooling treatment similar to that discussed above regarding the better aging treatment screen 166 configured to provide runtime information to the user regarding the running cooling treatment.

At an end of the skin clearing treatment, e.g., after about 11 minutes have elapsed, the display 154c is configured to show a skin clearing end screen. The skin clearing end screen is configured and used similar to the better aging end screen 168 discussed above, e.g., provide post-treatment information to the user regarding the skin clearing treatment that was just completed. The skin clearing end screen is configured to allow the user to go back to the main menu screen 160, e.g., by pushing the third control 154f down for less than the predetermined amount of time.

The skin sustain option allows selection of a skin sustain mode, which is a skin maintenance mode for maintaining smooth, even skin that glows, as indicated on the routines screen 162 (see FIG. 20) to help a user understand why this option may be desirable to the user. The skin sustain mode is for use after a certain amount of other light therapy treatment sessions have been completed to help maintain the beneficial therapeutic effect of the prior light therapy treatment sessions. As discussed herein, the control unit 154 is configured to keep track, e.g., in a memory at the PCB 154b, of a number of completed light therapy treatment sessions and to provide the number of completed light therapy treatment sessions to the user, e.g., via the display 154c. The skin sustain mode is configured to run for about eleven minutes, as also indicated on the routines screen 162 (see FIG. 20). A user is thus aware how long the skin sustain light therapy will last and thus how long the mask 100 must be worn for this treatment session.

In other implementations, the skin sustain mode is configured to run for a different amount of time, such as no longer than any of the light therapy treatment modes, e.g., than either of the skin clearing mode and the better aging mode, which may signal to the user that the treatment is being maintained and/or may help encourage the user to continue using to mask 100 daily for light therapy. For example, the sustain mode can be configured to run for no more than half the time of either the better aging mode or the skin clearing mode, e.g., about 4 minute sustain mode, about 11 minute better aging mode, and about 11 minute skin clearing mode. Being no more than half as long, a user will typically be able to tell that the sustain mode is shorter than the better aging mode and the skin clearing mode. For another example, the sustain mode can be configured to run no more than ⅔ of the shortest of the better aging mode or the skin clearing mode, e.g., about 4 minute sustain mode, about 6 minute better aging mode, and about 8 minute skin clearing mode. Being no more than ⅔ as long, a user will typically be able to tell that the sustain mode is shorter than the better aging mode and the skin clearing mode. Less of a difference may become difficult for the user to detect.

Similarly, the skin sustain mode can also be configured to run no longer than any of the cooling therapy modes, such as with an about 4 minute sustain mode and the shortest cooling therapy available being about 5 minutes.

The certain amount of other light therapy treatment sessions that have been completed before the skin sustain option allows enough time to elapse for the therapeutic benefits of the light therapy treatment sessions to have been realized. Without being maintained, the therapeutic benefits decrease over time, which may be frustrating for a user. The skin sustain mode may prevent user frustration by helping to maintain the therapeutic benefits of the light therapy treatment sessions that have been completed. In an exemplary implementation, the certain amount of other light therapy treatment sessions that have been completed is based on completion of a certain number of sessions for one skin treatment mode, e.g., the skin clearing mode or the better aging mode. In another exemplary implementation, the certain amount of other light therapy treatment sessions that have been completed is based on completion of a certain number of sessions for both of the skin treatment mode, e.g., a total number of completed light therapy treatment sessions whether in the skin clearing mode or the better aging mode.

Light therapy typically takes at least four weeks, and in some instances can take six to eight weeks or more, of daily mask use to provide a physical effect noticeable to the user. The certain amount of time can thus be, for example, an amount of time in a range of 4 to 10 weeks (corresponding to 28 to 70 daily treatments), in a range of 4 to 8 weeks (corresponding to 28 to 56 daily treatments), or in a range of 6 to 8 weeks (corresponding to 42 to 56 daily treatments), such as 4 weeks, 5 weeks, 6 weeks, 7 weeks, 8 weeks, 9 weeks, 10 weeks, or 12 weeks.

In some implementations, the control unit 154 is configured to lock out access to the skin sustain mode until the certain number of sessions has been completed, which may help a user achieve optimal treatment results. As discussed herein, the control unit 154 is configured to maintain a record of a number of completed treatment sessions.

In the skin sustain mode, the lights 124 of the mask 100 emit all three available types of light (red, blue, and IR) simultaneously. The power density of the red light emitted in the skin sustain mode is less than the power density of the red light emitted in the mask's other light therapy modes (better aging mode and skin clearing mode). As mentioned above, red light is configured to aid in anti-aging and penetrate deeper into skin than blue light and less deep than IR light, e.g., blue light penetrates into the epidermis, red light penetrates a first depth into the dermis, and IR light penetrates a second, greater depth into the dermis. By the red light having a lower power density in the skin sustain mode than in the mask's other light therapy modes, the skin sustain mode is configured to provide an anti-aging effect via the red light but not as aggressively as in the mask's other light therapy modes since the user has already completed enough treatment sessions in the mask's other light therapy modes to achieve therapeutic benefit from red light. In an exemplary implementation, a power density of the red light in the skin sustain mode is in a range of about 15% to about 35% of a power density of the right light in any of the mask's other light therapy modes, e.g., is in a range of about 20% to about 35%, is in a range of about 20% to about 30%, is in a range of about 20% to about 25%, is in a range of about 25% to about 30%, is in a range of about 23% to about 27%, is in a range of about 22% to about 24%, is in a range of about 25% to about 27%, is in a range of about 20% to about 25%, is about 20%, is about 21%, is about 22%, is about 23%, is about 24%, is about 25%, is about 26%, is about 27%, is about 28%, is about 29%, is about 30%, etc.

The power density of the IR light emitted in the skin sustain mode is less than the power density of the IR light emitted in the mask's other light therapy modes (better aging mode and skin clearing mode). As mentioned above, IR light is configured to aid in reducing deep wrinkles, preventing future fine lines, increasing collagen, and/or expanding blood vessels to allow for better blood flow. By the IR light having a lower power density in the skin sustain mode than in the mask's other light therapy modes, the skin sustain mode is configured to aid in reducing deep wrinkles, preventing future fine lines, increasing collagen, and/or expanding blood vessels to allow for better blood flow via the IR light but not as aggressively as in the mask's other light therapy modes since the user has already completed enough treatment sessions in the mask's other light therapy modes to achieve therapeutic benefit from IR light. In an exemplary implementation, a power density of the IR light in the skin sustain mode is in a range of about 70% to about 95% of a power density of the IR light in any of the mask's other light therapy modes, e.g., is in a range of about 75% to about 95%, is in a range of about 80% to about 95%, is in a range of about 80% to about 94%, is in a range of about 80% to about 93%, is in a range of about 80% to about 92%, is in a range of about 80% to about 91%, is in a range of about 80% to about 90%, is in a range of about 75% to about 85%, is in a range of about 90% to about 95%, is in a range of about 92% to about 94%, is about 80%, is about 83%, is about 85%, is about 90%, is about 93%, is about 95%, etc.

The power density of the blue light emitted in the skin sustain mode is less than the power density of the blue light emitted in at least a portion of the mask's skin clearing mode. In other words, the power density of the blue light emitted in the skin sustain mode is less than a maximum power density of the blue light emitted in the mask's skin clearing mode. As mentioned above, blue light is configured to affect the skin's surface. By the blue light having a lower power density in the skin sustain mode than the maximum power density of the blue light in the mask's skin clearing mode, the skin sustain mode is configured to affect the skin's surface via the blue light but not as aggressively as during at least a portion of the mask's skin clearing mode since the user has already completed enough treatment sessions in the mask's other light therapy modes to achieve therapeutic benefit from blue light. In an exemplary implementation, a power density of the blue light in the skin sustain mode is in a range of about 85% to about 95% of a maximum power density of the blue light in the mask's skin clearing mode, e.g., is in a range of about 90% to about 95%, is in a range of about 88% to about 94%, is in a range of about 91% to about 95%, is in a range of about 92% to about 92%, is in a range of about 93% to about 95%, is in a range of about 93% to about 94%, is in a range of about 85% to about 90%, is about 90%, is about 91%, is about 92%, is about 93%, is about 94%, is about 95%, etc.

In an exemplary implementation, a power density ratio of the red light to the IR light to the blue light is substantially 0.3:0.85:1, e.g., about 13% red light, about 40% IR light, and about 47% blue light, and the power density of a single light 124 in the skin sustain mode is about 17.0 mW/cm² for red light, about 59.8 mW/cm² for blue light, and about 51.2 mW/cm² for IR light for a total power density of each of the lights 124 in the better aging mode is thus about 128 mW/cm² and a total energy directed into the user face being about 39.1 J for red light, about 137.4 J for blue light, and about 117.6 J for IR light for a total energy directed into the user face for thermal purposes being about 156.7 J and for a total energy directed into the user face for anti-bacteria purposes being about 137.4 J. In this example, the power density of the red light emitted in the skin sustain mode is less than the power density of the red light emitted in the above examples of the mask's other light therapy modes, e.g., 17.0 mW/cm² is less than 64.0 mW/cm² (better aging mode example) and is less than 73.0 mW/cm² (skin clearing mode example), the power density of the IR light emitted in the skin sustain mode is less than the power density of the IR light emitted in the above examples of the mask's other light therapy modes, e.g., 51.2 mW/cm² is less than 64.0 mW/cm² (better aging mode example and skin clearing mode first stage example) and is less than 55.0 mW/cm² (skin clearing mode third stage example), and the power density of the blue light emitted in the skin sustain mode is less than the maximum power density of the blue light emitted in the above example of the mask's skin clearing mode, e.g., 59.8 mW/cm² is less than 64.0 mW/cm² (skin clearing mode first stage example).

Selection of the skin sustain option via the routines screen 162, e.g., by pushing the third control 154f down for less than the predetermined amount of time with the skin sustain option shown on the routines screen 162 as in FIG. 20, is configured to show a skin sustain start screen on the display 154c. The skin sustain start screen is configured and used similar to the better aging start screen 164 discussed above. When the user is ready to begin the skin sustain treatment, the user selects to start the skin sustain mode via the skin sustain start screen.

Starting the skin sustain mode is configured to cause a skin sustain treatment screen to be shown on the display 154c. The skin sustain treatment screen is configured and used similar to the better aging treatment screen 166 discussed above, e.g., provide runtime information to the user regarding the running skin sustain treatment.

Cooling therapy is configured to begin automatically with the start of the skin sustain treatment, similar to that discussed above regarding the better aging treatment. The skin sustain treatment screen is configured to provide runtime information to the user regarding the running cooling treatment similar to that discussed above regarding the better aging treatment screen 166 configured to provide runtime information to the user regarding the running cooling treatment.

At an end of the skin sustain treatment, e.g., after about 11 minutes have elapsed, the display 154c is configured to show a skin sustain end screen. The skin sustain end screen is configured and used similar to the better aging end screen 168 discussed above, e.g., provide post-treatment information to the user regarding the skin sustain treatment that was just completed. The skin sustain end screen is configured to allow the user to go back to the main menu screen 160, e.g., by pushing the third control 154f down for less than the predetermined amount of time.

The under-eye revive option allows selection of a under-eye revive mode, which allows selection of a cooling mode for eye depuffing without light therapy being provided simultaneous with the cooling therapy, as indicated on the routines screen 162 (see FIG. 21) to help a user understand why this option may be desirable to the user. The under-eye revive mode is configured to run for a time in a range of about five to fifteen minutes, as also indicated on the routines screen 162 (see FIG. 21). A specific cooling therapy run time is not provided on the routines screen 12 because the user selects the run time.

Selection of the under-eye revive option via the routines screen 162, e.g., by pushing the third control 154f down for less than the predetermined amount of time with the under-eye revive option shown on the routines screen 162 as in FIG. 21, is configured to show a cooling therapy duration selection screen on the display 154c. FIGS. 25 and 26 illustrate one implementation of a cooling therapy duration selection screen 170. Three cooling therapy durations are available in this illustrated implementation: 5 minutes, 10 minutes, and 15 minutes. FIG. 25 shows the 10 minute option selected. FIG. 26 shows the 15 minute option selected. All of the cooling therapy duration options are visible on the cooling therapy duration selection screen 170 in this illustrated implementation. If all of the cooling therapy duration options are not visible simultaneously, the user can scroll through the cooling therapy duration options, e.g., by using the third control 154f, to see all the available cooling therapy duration options.

In an exemplary implementation, the control unit 154 is configured to allow a user to select one of a plurality of cooling modes, e.g., by using the third control 154f to scroll through available cooling modes shown on the display 154c and select a desired one of the cooling modes. As mentioned above, each of the plurality of cooling modes is associated with a predetermined temperature to be applied to the user's skin via the first set of pads (and, at the user's option, also the second set of pads). The predetermined temperature for each of the plurality of cooling modes is stored in a memory at the PCB 154b of the control unit 154 and/or in a memory at the PCB 148 of the mask 100.

In an exemplary implementation, the plurality of cooling modes are each associated with a different predetermined temperature that is 25° C. or less and that is at least 3° C. above/below adjacent mode temperatures. Temperatures less than 25° C. will cause vasoconstriction that results in depuffing. A temperature difference of at least 3° C. will typically be felt by a user whereas a lower temperature difference will not typically be felt by a user, so having temperatures of cooling modes each being at least 3° C. apart will allow the user to feel a difference between each of the cooling modes and thus help the user choose a comfortable cooling mode and/or know that the cooling modes are different from one another. For example, the plurality of cooling modes can include first, second, and third cooling modes each associated with a different predetermined temperature, e.g., 16° C., 19° C., and 22° C. For another example, the plurality of cooling modes can include first, second, third, and fourth cooling modes each associated with a different predetermined temperature, e.g., 16° C., 19° C., 22° C., and 25° C. For another example, the plurality of cooling modes can include first, second, and third cooling modes each associated with a different predetermined temperature, e.g., 19° C., 22° C., and 25° C. For another example, the plurality of cooling modes can include first, second, and third cooling modes each associated with a different predetermined temperature, e.g., 15° C., 19° C., and 23° C. For another example, the plurality of cooling modes can include first and second cooling modes each associated with a different predetermined temperature, e.g., 16° C. and 22° C. For another example, the plurality of cooling modes can include first and second cooling modes each associated with a different predetermined temperature, e.g., 19° C. and 22° C. For another example, the plurality of cooling modes can include first and second cooling modes each associated with a different predetermined temperature, e.g., 16° C. and 19° C. For another example, the plurality of cooling modes can include first and second cooling modes each associated with a different predetermined temperature, e.g., 19° C. and 25° C. For another example, the plurality of cooling modes can include first and second cooling modes each associated with a different predetermined temperature. e.g., 15° C. and 19° C. For another example, the plurality of cooling modes can include first and second cooling modes each associated with a different predetermined temperature, e.g., 19° C. and 22° C. For another example, the plurality of cooling modes can include first and second cooling modes each associated with a different temperature in a range of 15° C. to 25° C.

The selected cooling mode can, in some implementations, as discussed above, be changed during the cooling therapy, which may provide the user greater control over cooling therapy.

In an exemplary implementation, whether or not the control unit 154 is configured to allow a user to select one of a plurality of cooling modes, the control unit 154 is configured to allow a user to select a time duration of cooling from among a plurality of time durations. In general, a longer duration of cooling may cause greater therapeutic effect and/or may help treat more stubborn problems. The plurality of time durations can be, for example, any two or more of five minutes, six minutes, seven minutes, eight minutes, nine minutes, ten minutes, twelve minutes, thirteen minutes, fourteen minutes, fifteen minutes, sixteen minutes, seventeen minutes, eighteen minutes, nineteen minutes, and twenty minutes. For example, the plurality of time durations can be five minutes apart from one another (e.g., five minutes, ten minutes, and fifteen minutes). A user will typically be able to tell differences between such staggered durations whereas shorter durations are typically more different to detect.

In instances in which a user chooses to receive light therapy and cooling therapy simultaneously, the duration of the cooling therapy is defined by a duration of the selected light therapy, as discussed above, instead of being independently selectable, which may ease user experience by not requiring the user to select multiple different times. Cooling therapy duration is not desirable to define light therapy duration since a maximum amount of time cooling therapy is provided is typically less than a maximum amount of time light therapy is provided, e.g., a maximum of fifteen minutes for cooling therapy and a maximum of eight minutes for light therapy.

When the user has scrolled to the desired cooling therapy duration option on the cooling therapy duration selection screen 170 and is ready to begin cooling therapy, the user selects to start the cooling therapy via the cooling therapy duration selection screen 170, e.g., push the third control 154f down for less than a predetermined amount of time. Starting the cooling therapy is configured to cause a cooling therapy screen to be shown on the display 154c. The cooling therapy screen is configured and used similar to the better aging treatment screen 166 discussed above, e.g., provide running cooling treatment information to the user regarding the running cooling therapy treatment.

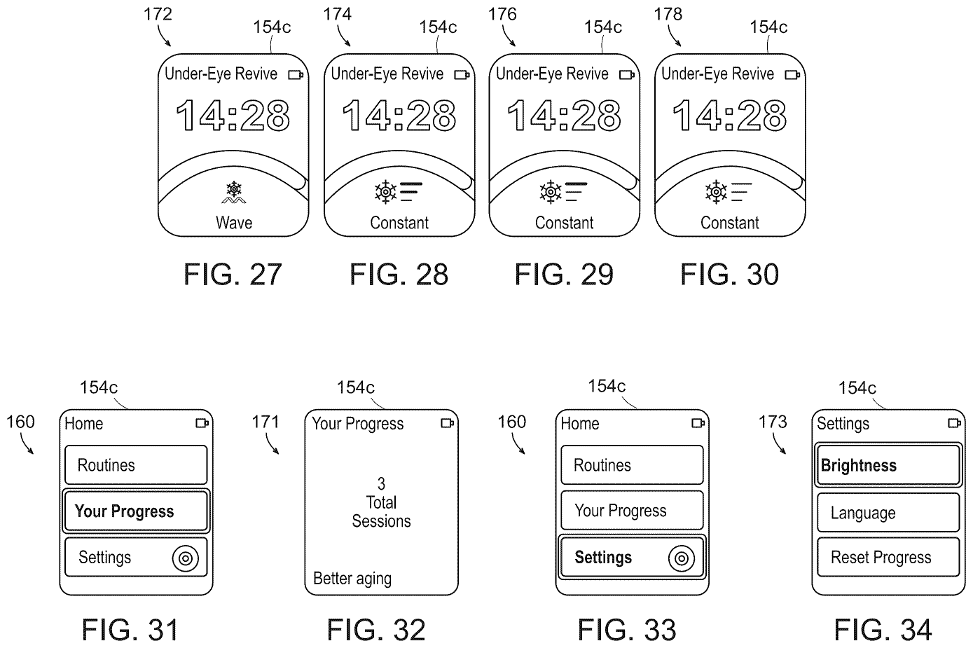

FIGS. 27-30 show implementations of the cooling therapy screen with different cooling treatment options selected. A fifteen minute duration has been selected for each of FIGS. 27-30. The cooling therapy screen 172 shown in FIG. 27 shows a time indicator for the running cooling therapy as a time countdown, shows a progress bar (an arced bar in this example), and shows the cooling mode as “wave” mode. No temperature level is shown because in wave mode, the cooling cycles through the different temperature levels. The cooling therapy screen 174 shown in FIG. 28 shows a time indicator for the running cooling therapy as a time countdown, shows a progress bar (an arced bar in this example), shows the cooling mode as “constant” mode, and shows the temperature level as a lowest level. The cooling therapy screen 176 shown in FIG. 29 shows a time indicator for the running cooling therapy as a time countdown, shows a progress bar (an arced bar in this example), shows the cooling mode as “constant” mode, and shows the temperature level as a middle level. The cooling therapy screen 178 shown in FIG. 30 shows a time indicator for the running cooling therapy as a time countdown, shows a progress bar (an arced bar in this example), shows the cooling mode as “constant” mode, and shows the temperature level as a highest level.

At any time during the cooling therapy, the user can choose, via the control unit 154 to change the temperature level or to change the cooling mode, as discussed above.

While cooling therapy is being provided, the control unit 154 is configured to maintain safe operating conditions. The control unit 154 is configured to compare the measured temperature of each of the first coolsinks 126a, 126b with a predetermined temperature associated with a current cooling mode, e.g., the lowest temperature level, the middle temperature level, or the highest temperature leave. If the measured temperature equals the predetermined temperature, then appropriate cooling is being provided to the user via the first coolsinks 126a, 126b (either directly or through the second coolsinks 128a, 128b). The control unit 154 takes no further action until a next measured temperature is received.

If the measured temperature does not equal the predetermined temperature for either of the first coolsinks 126a, 126b, then appropriate cooling is not being provided to the user via that coolsink. In response to the measured temperature being greater than the predetermined temperature, the control unit 154 is configured to trigger a corrective action to reduce temperature of the first coolsink with the out of range measured temperature. For example, if the first coolsink 126a has the out of range measured temperature, the control unit 154 can cause the first fan 130 to increase power to blow more air across the heatsink 134 to dissipate more heat, and/or the control unit 154 can cause less voltage to be provided to the first coolsink 126a to reduce temperature of the first coolsink 126a. In response to the measured temperature being less than the predetermined temperature, the control unit 154 is configured to trigger a corrective action to increase temperature of the first coolsink with the out of range measured temperature. For example, if the first coolsink 126a has the out of range measured temperature, the control unit 154 can cause the first fan 130 to decrease power to blow more air across the heatsink 134 to dissipate less heat and/or the control unit 154 can cause more voltage to be provided to the first coolsink 126a to increase temperature of the first coolsink 126a.

Determining if the measured temperature equals the predetermined temperature is, in some implementations, an exact match and is, in other implementations, a match within a +/− margin of acceptable tolerance.

In an exemplary implementation, the control unit 154, e.g., the PCB 154b of the control unit 154, is configured to receive temperature data measured by the temperature sensor 146 (and similar temperature sensor for the second cooling system) on a regular, periodic basis throughout the mask 100 providing cooling therapy. The control unit 154 may thus be able to cause corrective action to be performed as needed throughout the cooling therapy.

In some implementations, instead of the control unit 154, e.g., the PCB 154b of the control unit 154, being configured to compare the measured temperature of the first coolsinks 126a, 126b with a predetermined temperature associated with a current cooling mode, the mask 100, e.g., the PCB 148 of the mask 100, is configured to perform the comparison. If the measured temperature equals the predetermined temperature, the PCB 148 of the mask 100 takes no further action until a next measured temperature is received. If the measured temperature does not equal the predetermined temperature, the PCB 148 of the mask 100 communicates a temperature error to the control unit 154, e.g., the PCB 154b of the control unit 154, so the control unit 154 can cause appropriate corrective action, as discussed above.

At an end of the skin sustain treatment, e.g., after the selected cooling therapy duration has passed, the display 154c is configured to show a cooling therapy end screen. The cooling therapy end screen is configured and used similar to the better aging end screen 168 discussed above, e.g., provide post-treatment information to the user regarding the cooling therapy treatment that was just completed. The cooling therapy end screen is configured to allow the user to go back to the main menu screen 160, e.g., by pushing the third control 154f down for less than the predetermined amount of time.

Referring again to the main menu screen 160 of FIG. 17, a progress screen is configured to be shown on the display 154c and to show the plurality of therapeutic modes in response to user selection of the progress option via the main menu screen 160, e.g., selection of “your progress.” FIG. 31 shows the progress option selected.

FIG. 32 illustrates one implementation of a progress screen 171. The progress screen 171 shows historical information related to treatments provided by the mask 100 and thus show information regarding a user's treatment progress. As in this illustrated implementation, the progress information can include a number of times the better aging mode has been completed. Three completed better aging treatment sessions are shown in the example of FIG. 31. The progress screen 171 is configured to allow the user to scroll through the plurality of therapeutic modes, e.g., by using the third control 154f, to see progress for each of the plurality of therapeutic modes.

Referring again to the main menu screen 160 of FIG. 17, a settings screen is configured to be shown on the display 154c and to show available operational settings in response to user selection of the settings option via the main menu screen 160, e.g., selection of “settings.” FIG. 33 shows the settings option selected.

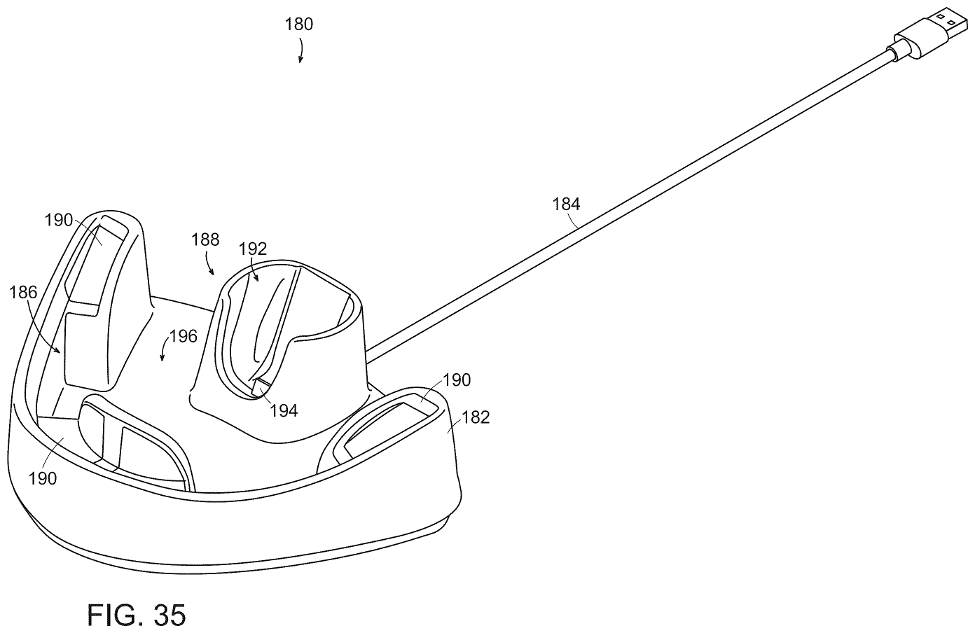

FIG. 34 illustrates one implementation of a settings screen 173. The settings screen 173 shows operational parameters that the user can customize. As in this illustrated implementation, the operational parameters can include a brightness of the display 154c, language used on the display 154c (e.g., English, Spanish, etc.), and progress reset to reset the user's treatment progress amount to zero. If all of the setting options are not visible simultaneously on the settings screen 173, as in this illustrated implementation, the user can scroll through the setting options, e.g., by using the third control 154f, to see all the available setting options. Examples of other operational parameters that can be adjustable via the settings screen 173 include tutorial information, technical support (which can be provided directly via the display 154c and/or by providing a QR code, a URL, or other direction to electronic help on the Internet), factory reset to reset all settings to factory defaults, color scheme of the display 154c, and contrast level of the display 154c.

As mentioned above, the control unit 154 of FIG. 1 includes a rechargeable power source 154a in this illustrated implementation. The power source 154a can be recharged in any of a variety of ways. In an exemplary implementation, the control unit 154 is configured to be docked in a charging stand for recharging. The charging stand also serves as a storage stand for the control unit 154 and the mask 100. Recharging may therefore occur conveniently at the same location where the control unit 154 and the mask 100 are being stored between uses. The charging stand can have a variety of configurations.

FIG. 35 illustrates one exemplary implementation of charging stand 180. The illustrated charging stand 180 is sized and shaped for use with the mask 100 and the control unit 154 of FIG. 1. Other charging stands having different sizes and/or shapes can be used similarly with other masks and control units.

The charging stand 180 includes a base 182 and a power cord 184. The power cord 184 is configured to operably connect to a power source to provide power for recharging. The power cord 184 in this illustrated implementation is a USB cord configured to be plugged into a USB port, but other types of power cords can be used, such as a cord configured to be plugged into a wall outlet.

The base 182 is configured to dock the mask 100 and the control unit 154. The base 182 includes a first docking area 186 configured to dock the mask 100 and a second docking area 188 configured to dock the control unit 154. The first docking area 186 includes one or more mask supports 190 configured to seat the mask 100. The one or more mask supports 190 are configured to prevent the mask 100 from tipping over or falling off the stand 180. The bottom of the mask 100 is configured to rest on the one or more mask supports 190 in this illustrated implementation. The mask supports 190 includes three support in this illustrated implementation but another number of mask supports 190 may be provided.

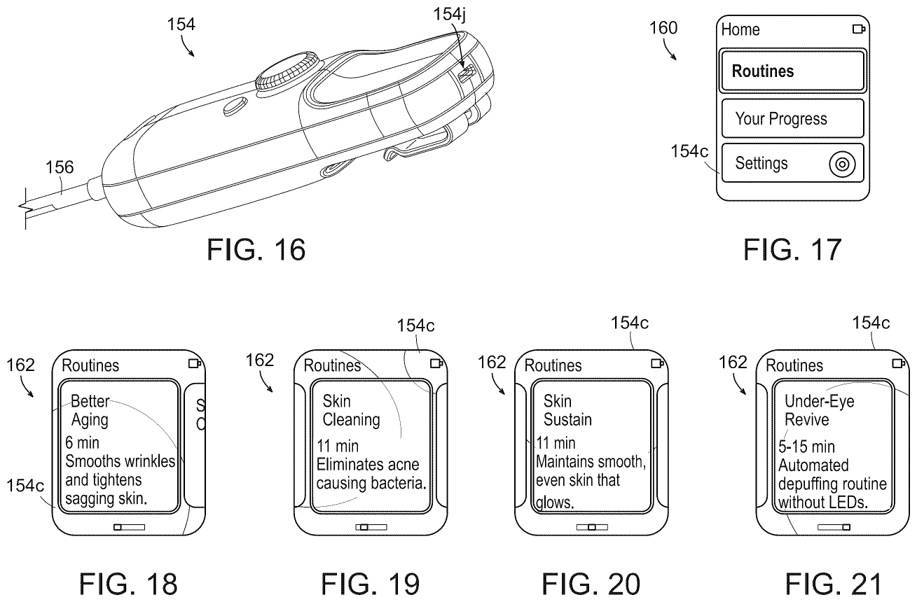

The second docking area 188 defines a cavity 192 configured to receive the control unit 154 therein. A charger interface 194 is located within the cavity 192. The charger interface 194 is configured to be received in a charging port 154j of the control unit 154 (see FIG. 16). With the power cord 184 attached to a power source, e.g., plugged into a USB port, and with the charger interface 194 received in the charging port 154j, power is configured to be provided to the control unit 154 via the connected charger interface 194 and charging port 154j to recharge the power source 154a of the control unit 154. Recharging is configured to occur automatically with the power cord 184 attached to a power source and with the charger interface 194 received in the charging port 154j, which may help ensure that recharging occurs regularly since the mask 100 and the control unit 154 will typically be stored in the stand 180 when the mask 100 is not being worn by a user. Alternatively, the charging stand 180 and/or the control unit 154 can include a recharging control configured to allow a user to turn on/off recharging.

The charging stand 180 includes a storage area 196 between the first and second docking areas 186, 188 that defines space configured to accommodate the cable 156 while the mask 100 and the control unit 154 are docked. A user may bend the cable 156 as needed to fit the cable 156 into the storage area 196.

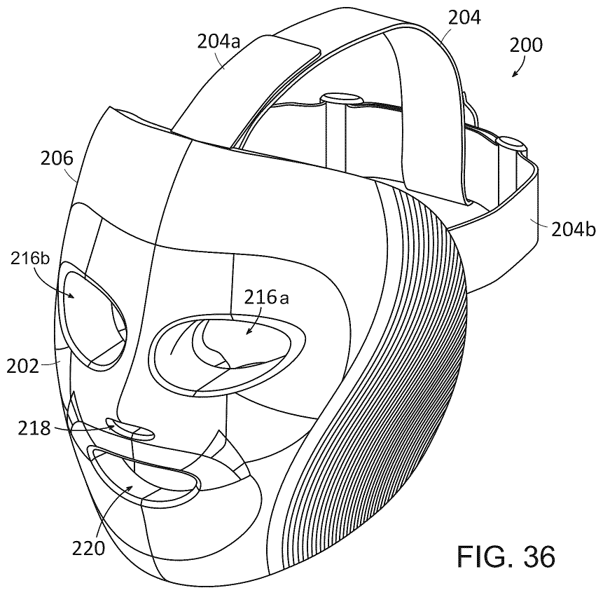

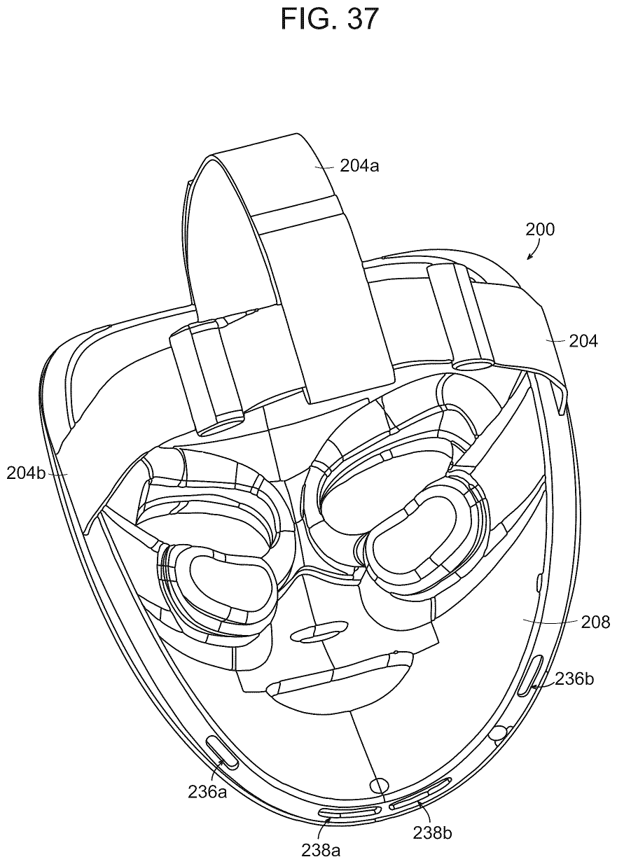

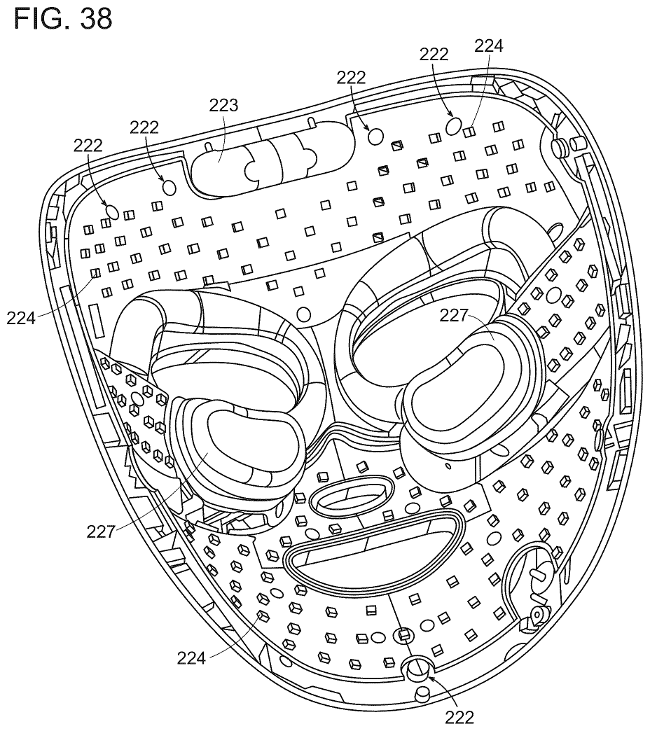

FIGS. 36 and 37 illustrate another implementation of a face mask 200 configured to provide cooling therapy to a user wearing the face mask 200. The face mask 200 in this illustrated implementation is also configured to provide light therapy.

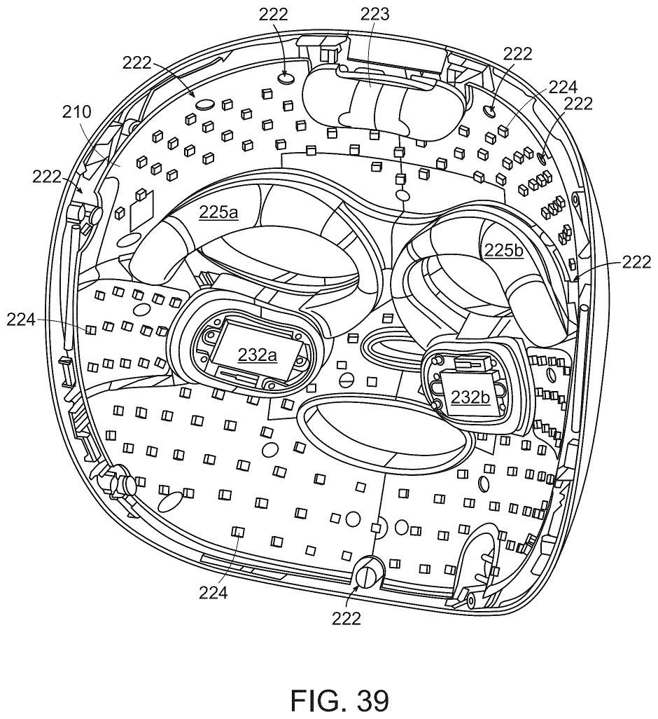

The face mask 200 of FIGS. 36 and 37 is generally configured and used similar to the face mask 100 of FIG. 1 and includes a base 202 including an outer shell 206 (see FIG. 36), an inner shell 208 (see FIG. 37), and an intermediate shell 210 (see FIGS. 38 and 39); a support 204 including first and second straps 204a, 204b; first and second air inflow paths (obscured in the figures), first and second air outflow paths (obscured in the figures); a plurality of openings including a first eye opening 216a, a second eye opening 216b, a nose opening 218, and a mouth opening 220; cut-outs 222 at a plurality of interior connection points; a light assembly including a plurality of lights 224; a PCB (obscured in the figures); first and second cooling systems each including a fan (obscured in the figures), a thermoelectric cooling device (first and second thermoelectric cooling devices 232a, 232b shown in FIG. 39), and a heat sink (obscured in the figures); a forehead pad 223; first and second eye shields 225a, 225b; a set of eye pads 227, first and second air inlets 236a, 236b; first and second air outlets 238a, 238b; and first and second noise attenuation systems (obscured in the figures). The mask 200 can include any one or more of the variations discussed above that the mask 100 of FIG. 1 may have.

FIG. 39 shows the mask 200 without the set of pads 227 attached to the mask 100 for purposes of illustration, since the set of pads 227 are non-removably attached to the mask 200 in this illustrated implementation. The set of pads 227 are the only set of pads in this illustrated embodiment.

A control unit (now shown) is configured to be operably coupled with the mask 200 using a cable (not shown) and is generally configured and used similar to the control unit 154 of FIG. 1. The control unit used with the mask 200 can include any one or more of the variations discussed above that the control unit 154 of FIG. 1 may have.

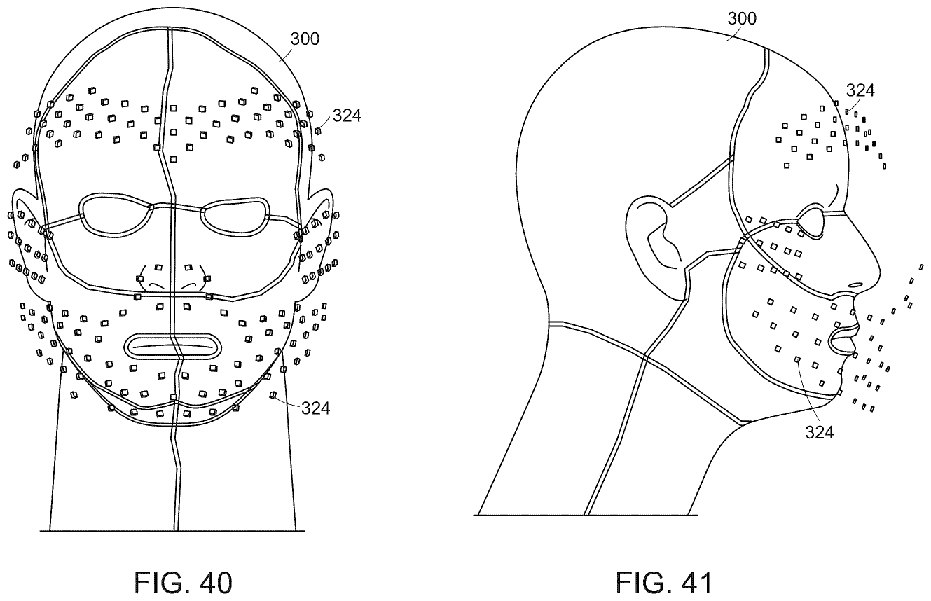

As mentioned above, the face masks described herein, e.g., the masks 100, 200 of FIGS. 1 and 36 and other masks, can have any number and any arrangement of lights configured to provide light therapy to a user wearing a mask. FIGS. 40 and 41 illustrate the number and arrangement of the lights 124, 224 of the masks 100, 200 of FIGS. 1 and 36, which may also be used for other masks described herein, as lights 324 of a face mask (the mask is not shown for clarity of illustration). A number of the lights 324 in FIGS. 40 and 41 is one hundred sixty. The lights 324 are shown relative to a model 300 of a head as if the face mask including the lights 324 is being worn on the head. The illustrated model 300 is a National Institute for Occupational Safety and Health (NIOSH) digital headform, size medium.

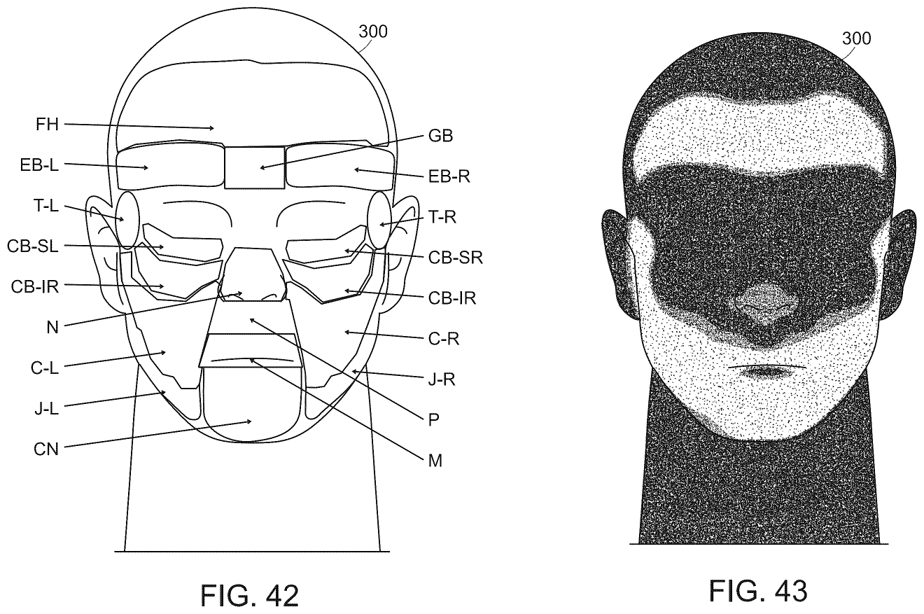

The number and the arrangement of the lights 324 of FIGS. 40 and 41 is optimized for uniformity and coverage of light emission across various facial regions shown in FIG. 42 and identified below in Table 1. The facial regions in FIG. 42 are shown with respect to the model 300 of FIGS. 40 and 41. The arrangement of the lights 324 refers to both a distance of the lights 324 from the user's face when the user is wearing the mask and a pattern of the lights 324 on the mask.

TABLE 1

| ID | Facial Region |
|---|---|
| FH | Forehead |
| EB-L | Eyebrow Left |
| EB-R | Eyebrow Right |
| GB | Glabella |
| T-L | Temple Left |
| T-R | Temple Right |
| CB-SL | Cheekbone Superior Left |
| CB-SR | Cheekbone Superior Right |
| CB-IL | Cheekbone Inferior Left |
| CB-IR | Cheekbone Inferior Right |
| N | Nose |
| C-L | Cheek Left |
| C-R | Cheek Right |
| J-L | Jaw Left |
| J-R | Jaw Right |
| CN | Chin |
| M | Mouth |
| P | Philtrum |

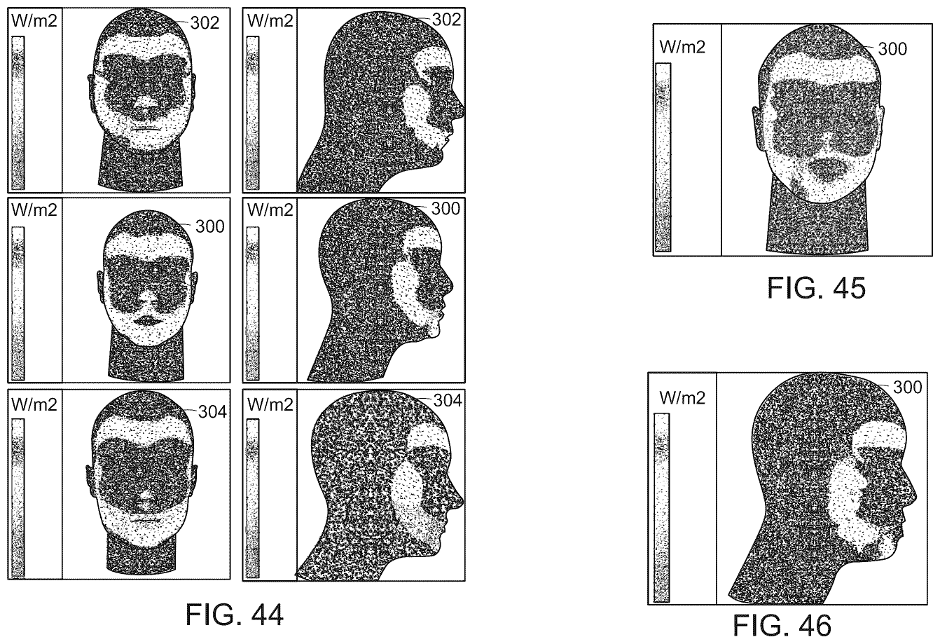

FIG. 43 shows one example of a light intensity distribution of the lights 324 on the model 300 as simulated with Speos optical simulation software. All of red light, and blue light, and IR light are being emitted by each of the lights 324 in the example of FIG. 43. FIG. 44 illustrates the light intensity distribution being substantially the same as in FIG. 43 for heads larger than the medium size model 300, as indicated by a light intensity distribution of the lights 324 on a NIOSH digital headform model 302, size large, and for heads larger than the medium size model 300, as indicated by a light intensity distribution of the lights 324 on a NIOSH digital headform model 304, size small. The power density of each of the lights 324 in the simulations of FIGS. 43 and 44 is 128 mW/cm².

As shown in FIGS. 42 and 43, the facial regions of Table 1 receive light emitted from the lights 324 except for select facial regions, shown as dark areas in FIGS. 42 and 43, that are covered by the mask but are protected from the emitted light by features of the mask. As shown in FIGS. 42 and 43, dark areas above the eyes in the EB-L and EB-R regions and to the sides of the eyes in the T-L and T-R regions demonstrate light blocking provided by eye shields of the mask configured to help protect eyes from light emitted from the lights 324, such as the first and second eye shields 152a, 152b of the mask 100 of FIG. 1 and the first and second eye shields 225a, 225b of the mask 200 of FIG. 36. Various exemplary implementations of shields of face masks configured to facilitate protecting a user's eyes from light emitted by lights of the face mask are described further in, for example, U.S. patent application Ser. No. 18/411,806 entitled “Light Emitting Face Masks” filed Jan. 12, 2024, which is hereby incorporated by reference in its entirety.

As shown in FIGS. 42 and 43, dark areas under the eyes in the CB-SL and CB-SR regions demonstrate light blocking provided by eye pads of the mask configured to help protect eyes from light emitted from the lights 324, such as the first set of pads of the mask 100 of FIG. 1, the second set of pads of the mask 100 of FIG. 1, and the set of pads of the mask 200 of FIG. 36. Various exemplary implementations of eye pads of face masks configured to facilitate protecting a user's eyes from light emitted by lights of the face mask are described further in, for example, U.S. patent application Ser. No. 18/411,644 entitled “Face Masks With Noise Attenuation” filed Jan. 12, 2024, which is hereby incorporated by reference in its entirety.

As shown in FIGS. 42 and 43, a dark, central indented area at a top of the FH region demonstrates light blocking provided by a forehead pad (also referred to herein as a “forehead bumper”) configured to help space the lights 324 from the user's face, such as the forehead pad 150 of the mask 100 of FIG. 1 and the forehead pad 223 of the mask 200 of FIG. 36.

As shown in FIGS. 40 and 41, all of the lights 324 of the mask are spaced a distance from the user's face. The eye shields, the forehead pad, and the eye pads of the mask are configured to cooperate together to help provide this distance when the mask is being worn by a user. The lights 324 being spaced from the user's face helps allow light emitted from the lights 324 to illuminate a greater area of the skin than if the lights 324 were closer to the user's face, thereby helping to provide greater uniformity and coverage. In some implementations, the mask includes only one or two of the eye shields, the forehead pad, and the eye pads.

Dimensions and curvature of the one, two, or three of the eye shields, the forehead pad, and the eye pads of the mask facilitate the maintenance of the distance of the lights 324 from the user's face so that there is a gap of space between each of the lights 324 and the user's face. Each of the eye shields, the forehead pad, and the eye pads has a minimum height for any portion thereof, with the minimum height corresponding to the distance between the one, two, or three of eye shields, the forehead pad, and the eye pads and the user's face. In an exemplary implementation, the minimum height is in a range between about 5 mm and about 50 mm, for example in a range between about 10 mm and about 40 mm or in a range between about 30 mm and about 40 mm. In such an implementation, with the minimum height being in a range between about 5 mm and about 50 mm, the distance between the plurality of lights 324 and the user's face is in a range between about 10 mm and about 40 mm. In implementations in which any of the one, two, or three of eye shields, the forehead pad, and the eye pads is formed of a compressible material, the minimum height can correspond to a compressed configuration, in which the mask 100 is being worn by a user and the compressible material is at least partially compressed, or an uncompressed configuration, in which the mask 100 is not being worn by a user.

In some implementations, the distance is the same for each of the plurality of lights 324. In other implementations, the distance of at least one light of the plurality of lights 324 is different from the distance of at least one other of the plurality of lights 324.

Tables 2 and 3 demonstrate that the number and the arrangement of the lights 324 of FIGS. 40 and 41 is optimized for uniformity and coverage of light emission when the mask is being worn by a user and is delivering light therapy to the user.

TABLE 2

| Irradiance & Uniformity | Weight | SS | SC | BA |
|---|---|---|---|---|
| FH | 4.66 | 0.5 | 0.5 | 0.5 |
| EB-L | 1.295 | 0.0 | 0.0 | 0.0 |
| EB-R | 1.295 | 0.0 | 0.0 | 0.0 |
| GB | 3.47 | 0.0 | 0.0 | 0.0 |
| T-L | 0.5 | 0.3 | 0.3 | 0.3 |
| T-R | 0.5 | 0.3 | 0.3 | 0.3 |
| CB-SL | 0.61 | 0.0 | 0.0 | 0.0 |
| CB-SR | 0.61 | 0.0 | 0.0 | 0.0 |
| CB-IL | 0.7575 | 0.0 | 0.0 | 0.0 |
| CB-IR | 0.7575 | 0.0 | 0.0 | 0.0 |
| N | 3.8 | 0.1 | 0.1 | 0.1 |
| C-L | 2.48 | 0.9 | 0.9 | 0.9 |
| C-R | 2.48 | 1.0 | 1.0 | 1.0 |
| J-L | 1.825 | 0.3 | 0.3 | 0.3 |
| J-R | 1.825 | 0.4 | 0.4 | 0.4 |
| P | 2.48 | 0.6 | 0.6 | 0.6 |
| M | 1 | 0.9 | 0.9 | 0.9 |
| CN | 4.5 | 1.0 | 1.0 | 1.0 |
| Score | — | 16.0 | 16.0 | 16.0 |
| Score Normalized | — | 1.00 | 1.00 | 1.00 |

TABLE 3

| Irradiance & Uniformity | Weight | SS | SC | BA |
|---|---|---|---|---|
| FH | 5.85 | 0.5 | 0.5 | 0.5 |
| EB-L | 1.415 | 0.0 | 0.0 | 0.0 |
| EB-R | 1.415 | 0.0 | 0.0 | 0.0 |
| GB | 5.49 | 0.0 | 0.0 | 0.0 |
| T-L | 2.62 | 0.3 | 0.3 | 0.3 |
| T-R | 2.62 | 0.3 | 0.3 | 0.3 |
| CB-SL | 1.37 | 0.0 | 0.0 | 0.0 |
| CB-SR | 1.37 | 0.0 | 0.0 | 0.0 |
| CB-IL | 1.0725 | 0.0 | 0.0 | 0.0 |
| CB-IR | 1.0725 | 0.0 | 0.0 | 0.0 |
| N | 1 | 0.1 | 0.1 | 0.1 |
| C-L | 1.77 | 0.9 | 0.9 | 0.9 |
| C-R | 1.77 | 1.0 | 1.0 | 1.0 |
| J-L | 1.265 | 0.3 | 0.3 | 0.3 |
| J-R | 1.265 | 0.4 | 0.4 | 0.4 |
| P | 1.35 | 0.6 | 0.6 | 0.6 |
| M | 1 | 0.9 | 0.9 | 0.9 |
| CN | 1.62 | 1.0 | 1.0 | 1.0 |
| Score | — | 12.4 | 12.4 | 12.4 |
| Score Normalized | — | 1.00 | 1.00 | 1.00 |

Table 2 shows one simulation of light irradiance and uniformity in each of the eighteen facial regions of FIG. 42 for each of the mask's light therapy modes: skin sustain (SS), skin clearing (SC), and better aging (BA). The weights in FIG. 42 are based on a perceived importance of the facial region in addressing acne using the light therapy, with a higher weight indicting a higher perceived importance of uniformity and coverage of light emission in that facial region to treat acne. Thus, the FH, CN, N, GB, C-L, C-R, and P facial regions have a highest weight in Table 2 as those are the facial regions where acne is most likely to be present or develop.

Table 3 shows another simulation of light irradiance and uniformity in each of the eighteen facial regions of FIG. 42 for each of the mask's light therapy modes: SS, SC, and BA. The weights in FIG. 42 are based on perceived importance of the facial region in addressing wrinkles using the light therapy, with a higher weight indicting a higher perceived importance of uniformity and coverage of light emission in that facial region to treat wrinkles. Thus, the FH, GB, T-L, and T-R facial regions have a highest weight in Table 2 as those are the facial regions where wrinkles most likely to be present or develop.

In each of the simulations of Tables 2 and 3, a metric was calculated as follows, with I_{average} being average irradiance in units of W/cm², α being a contrast value that is the standard deviation of the irradiance within the facial region divided by the average value (i.e., a facial region that has uniform received intensity would have α=0), and σ being the standard deviation of the irradiance across the facial region:

$$Metric = \frac{{I}_{a v e r a g e}}{α} = \frac{{I}_{a v e r a g e}^{2}}{σ}$$

The metric indicates that, for the same average irradiance in a given facial region, a mask that offers more uniform illumination will perform better. Similarly, for the same variation in intensity across the facial region (i.e., same α), a mask with average higher intensity will perform better. In this way, the metric promotes higher average intensities and more uniform coverage.

In the simulation, the metric for each facial region in each of the SS, SC, and BA modes was normalized to a range of 0 to 1 (worst to best) and then multiplied by the weight factor for that facial region. The resulting score for each facial region in each of the SS, SC, and BA modes is shown in Tables 2 and 3. As shown in Tables 2 and 3, the FH, CN, C-L, C-R, P, J-R, J-L, T-L, T-R, and N facial regions have the highest (best) scores in all of the SS, SC, and BA modes.

The masks described herein are intended to be positioned substantially the same relative to each particular user's face and include physical features configured to help such conformity. The physical features include, for example, a support (e.g., the support 104 of the mask 100 of FIG. 1 and the support 204 of the mask 200 of FIG. 36), a forehead bumper (e.g., the forehead pad 150 of the mask 100 of FIG. 1 and the forehead pad 223 of the mask 200 of FIG. 36), eye shields (e.g., the first and second eye shields 152a, 152b of the mask 100 of FIG. 1 the first and second eye shields 225a, 225b of the mask 200 of FIG. 36), and eye pads (e.g., the first set of pads of the mask 100 of FIG. 1, the second set of pads of the mask 100 of FIG. 1, and the set of pads of the mask 200 of FIG. 36). However, in some atypical use situations, the mask may be misaligned relative to a user's face. FIGS. 45 and 46 demonstrate that, even with a gross misalignment of the mask relative to the model 300, the number and arrangement of the lights 324 provide substantially the same irradiation pattern as for a normally positioned mask as represented by FIGS. 43 and 44.

The subject matter described herein can be implemented in analog electronic circuitry, digital electronic circuitry, and/or in computer software, firmware, or hardware, including the structural means disclosed in this specification and structural equivalents thereof or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, algorithm, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code).

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices). The processor and the memory can be supplemented by, or incorporated in, special purpose logic circuitry.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor-readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor). The modules described herein can be combined, integrated, separated, and/or duplicated to support various applications. Also, a function described herein as being performed at a particular module can be performed at one or more other modules and/or by one or more other devices instead of or in addition to the function performed at the particular module.

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A therapeutic device, comprising:
a face mask configured to be worn on a face of a user, the face mask comprising a plurality of light emitting diodes (LEDs) configured to emit light toward and to skin on the face of the user with the user wearing the face mask;
wherein each of the plurality of LEDs is configured to emit first light having a first wavelength, second light having a second wavelength, and third light having a third wavelength;
the face mask is configured to operate selectively in a plurality of light therapy modes;
in a first light therapy mode of the plurality of light therapy modes, each of the LEDs is configured to emit the second light at a first power density, to emit the first light at a second power density, and to emit the third light at a third power density;
in a second light therapy mode of the plurality of light therapy modes, each of the LEDs is configured to emit the second light at a fourth power density, to emit the first light at a fifth power density, and to emit the third light at a sixth power density; and
in a third light therapy mode of the plurality of light therapy modes, each of the LEDs is configured to emit the first light, to emit the second light having a seventh power density that is less than each of the first and fourth power densities, and to emit the third light at an eighth power density that is less than each of the third and sixth densities.

2. The therapeutic device of claim 1, wherein the seventh power density is in a range of about 15% to about 35% of each of the first and fourth power densities; and
the eighth power density is in a range of about 70% to about 95% of each of the third and sixth power densities.

3. The therapeutic device of claim 1, wherein the third light therapy mode is configured to be selected only after at least one of the first and second light therapy modes has been run a predetermined number of times.

4. The therapeutic device of claim 1, wherein in the third light therapy mode, each of the LEDs is configured to emit the first light at a ninth power density that is less than a maximum of the fifth power density.

5. The therapeutic device of claim 4, wherein the ninth power density is in a range of about 85% to about 95% the maximum of the fifth power density.

6. The therapeutic device of claim 1, wherein the first light is blue light, the second light is red light, and the third light is infrared (IR) light.

7. The therapeutic device of claim 1, wherein the first wavelength is in a range of 450 nm to 495 nm, the second wavelength is in a range of 620 nm to 740 nm, and the third wavelength is in a range of 800 nm to 2500 nm.

8. The therapeutic device of claim 1, wherein a total power density of each of the lights in each of the first, second, and third light therapy modes is about 128 mW/cm².

9. The therapeutic device of claim 6, wherein a total number of plurality lights is 160.

10. The therapeutic device of claim 1, wherein each of the lights is configured to be positioned a distance from the face of the user with the user wearing the face mask.

11. The therapeutic device of claim 8, wherein the distance is in a range between about 10 mm and about 40 mm.

12. The therapeutic device of claim 8, wherein the distance of each LED of the plurality of LEDs is substantially equal for each LED.

13. The therapeutic device of claim 8, wherein the distance of at least one LED of the plurality of LED is different from the distance of at least one other of the plurality of LEDs.

14. The therapeutic device of claim 8, wherein the face mask further comprises a spacer configured to contact the face of the user with the user wearing the face mask, the spacer comprising at least one of:
a first spacer configured to contact a forehead of the face of the user,
a second spacer configured to contact skin under eyes of the user, and
a third spacer configured to contact skin over the eyes of the user.

15. The therapeutic device of claim 12, wherein the face mask includes at least one of the second spacer and the third spacer; and
the at least one of the second spacer and the third spacer is configured to block the light emitted by the plurality of LEDs from reaching the eyes of the user.

16. The therapeutic device of claim 1, wherein the face mask further comprises:
a first thermoelectric cooling device configured to be positioned under a first eye of the user and configured to generate cooling configured to be applied to skin under the first eye of the user, and
a second thermoelectric cooling device configured to be positioned under a second eye of the user and configured to generate cooling configured to be applied to skin under the second eye of the user.

17. The therapeutic device of claim 14, wherein the plurality of LEDs are configured to emit the light simultaneously with the first and second thermoelectric cooling devices generating the cooling.

18. The therapeutic device of claim 14, wherein the plurality of LEDs are configured to emit the light without the first and second thermoelectric cooling devices generating the cooling; and
the first and second thermoelectric cooling devices are configured to generate the cooling without the plurality of LEDs emitting the light.
